(19) United States
(12) Patent Application Publication
(10) Pub. No.: US 2026/0131166 A1
(43) Pub. Date: May 14, 2026

(54) RADIATION THERAPY SYSTEM WITH O-RING LINAC AND ROTATABLE TREATMENT COUCH

(71) Applicant: VARIAN MEDICAL SYSTEMS, INC., Palo Alto, CA (US)

(72) Inventors: Ross HANNIBAL, Saratoga, CA (US); Reto FILIBERTI, Baar (CH); Stefan WEIRICH, Schlieren (CH); Mika MIETTINEN, San Francisco, CA (US); Karl BUSH, Palo Alto, CA (US); John VAN HETEREN, Foster City, CA (US)

(73) Assignee: VARIAN MEDICAL SYSTEMS, INC., Palo Alto, CA (US)

(21) Appl. No.: 18/946,970

(22) Filed: Nov. 14, 2024

(51) Int. Cl. A61N 5/10

(52) U.S. Cl. CPC A61N 5/1081; A61N 5/1049; A61N 2005/1061; A61N 2005/1087

(57) ABSTRACT

An example radiation therapy system is described. The system includes a stationary support structure fixed to a support surface, a rotatable gantry with a treatment-delivering radiation source and at least one X-ray imager mounted thereon, and a couch-positioning system. The treatment-delivering radiation source directs a treatment beam through a treatment isocenter of the radiation therapy system. The rotatable gantry is rotatably coupled to the support structure and is configured to rotate about a bore of the radiation therapy system. The couch-positioning system is rotatably coupled to the support surface and the center of rotation is vertically aligned with the treatment isocenter.

### CROSS-REFERENCE TO RELATED APPLICATIONS

The present application is related to co-pending U.S. patent application Ser. No. ______, filed Nov. 14, 2024 (124-0072-US2), U.S. patent application Ser. No. ______, filed Nov. 14, 2024 (124-0072-US3), U.S. patent application Ser. No. ______, filed Nov. 14, 2024 (124-0072-US4), U.S. patent application Ser. No. ______, filed Nov. 14, 2024 (124-0072-US5), and U.S. patent application Ser. No. ______, filed Nov. 14, 2024 (124-0072-US6). The aforementioned U.S. patent applications are incorporated herein by reference.

### BACKGROUND

Unless otherwise indicated herein, the approaches described in this section are not prior art to the claims in this application and are not admitted to be prior art by inclusion in this section.

Radiation therapy (also called radiotherapy) is a cancer treatment that employs high doses of ionizing radiation to kill cancer cells, such as X-rays or high-energy electrons, protons, or other heavy charged particles. Generally, radiation therapy is a localized treatment for a specific target tissue, such as a cancerous tumor. Ideally, radiation therapy is performed on a planning target volume (i.e., the target tissue) that spares the surrounding normal tissue from receiving doses above specified tolerances, thereby minimizing the risk of damage to healthy tissue. For example, to accurately supply a planned radiation dose, the spatial distribution of delivered radiation dose within the patient must closely match the spatial distribution of the planned radiation dose. So that the planned radiation dose is correctly supplied to the planning target volume during radiation therapy, the patient should be correctly positioned relative to the radiation source that provides the radiation therapy. In addition, precisely controlling the position of the radiation source relative to the patient is a significant factor in accurately targeting tissue in the patient. In light of the above, various system architectures have been developed that enable precise and repeatable positioning of a patient relative to a treatment isocenter of the system and precise and repeatable rotational positioning of a radiation source about the treatment isocenter. The most commonly employed radiation therapy systems have either an O-ring linear accelerator (LINAC) architecture or a C-arm LINAC architecture.

In radiation therapy systems that include an O-ring LINAC, the LINAC and associated imaging systems are rotated about the treatment isocenter via a circular gantry that is centered on the isocenter. The circular gantry is rotatably coupled to a support structure and rotates about a fixed cylindrical bore in which a patient is positioned. In such systems, the accuracy of the radiation dose received by the patient is determined in part by deflections of the gantry that occur during rotation. Because the support structure can be built around the bore and positioned in a vertical plane close to the isocenter, the LINAC and associated imaging systems are not cantilevered away from the support structure a large distance. As a result, deflections of the gantry, LINAC, and imaging systems in an O-ring LINAC are more easily controlled and deformation of the gantry under load is reduced compared to other radiation therapy systems. Further, in O-ring LINACS, the patient is protected from collisions with rotating parts while positioned in the bore, for example by a housing or other protective cover. Consequently, with no collision threat for the patient, the rotational speed of the gantry in an O-ring LINAC is not limited by safety concerns.

In radiation therapy systems that include a C-arm LINAC, the LINAC and associated imaging systems are mounted on a C-arm that is rotated about the treatment isocenter. Because the isocenter is not enclosed by a bore, in many instances, the patient couch of the system can be rotated relative to the treatment isocenter. As a result, the orientation of the patient to the plane of rotation of the LINAC and the imagers enables greater versatility of both treatment and imaging. A C-arm LINAC can be particularly useful for intracranial treatments, which generally cannot be performed with as high a dose fall-off gradient on an O-ring LINAC.

### SUMMARY

According to some embodiments, a radiation therapy system includes a stationary support structure fixed to a support surface external to the radiation therapy system, a rotatable gantry with a treatment-delivering radiation source and at least one X-ray imager mounted thereon, and a couch-positioning system for rotating a couch about a center of rotation. The treatment-delivering radiation source directs a treatment beam through a treatment isocenter of the radiation therapy system, and the rotatable gantry is rotatably coupled to the support structure and is configured to rotate about a bore of the radiation therapy system. The couch-positioning system is rotatably coupled to the support surface and the center of rotation is vertically aligned with the treatment isocenter.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. These drawings depict only several embodiments in accordance with the disclosure and are, therefore, not to be considered limiting of its scope. The disclosure will be described with additional specificity and detail through use of the accompanying drawings.

FIG. 1 is a perspective view of a radiation therapy system, according to various embodiments.

FIG. 2 is a perspective view of the therapy system of FIG. 1, according to various embodiments.

FIG. 3A schematically illustrates a side view of the therapy system of FIG. 1, according to various embodiments

FIG. 3B schematically illustrates a plan view of the therapy system of FIG. 1, according to various embodiments.

FIG. 4 schematically illustrates a plan view of a funnel-shaped cavity of a radiation therapy system with a couch positioned therein, according to various embodiments.

FIG. 5 is a perspective view of a radiation therapy system with angled X-ray imagers, according to various embodiments.

FIG. 6 is a perspective view of the radiation therapy system of FIG. 5 with X-ray imagers receiving X-rays, according to various embodiments.

FIG. 7 schematically illustrates a side view of the radiation therapy system of FIG. 5, according to various embodiments.

FIG. 8 is a schematic perspective view of an X-ray imager of a radiotherapy system that has a curved configuration, according to various embodiments.

FIG. 9 schematically illustrates a side view of a radiation therapy system with a rotational mechanism for positioning an X-ray imager, according to various embodiments.

FIG. 10 schematically illustrates a side view of a radiation therapy system with a dual rotary-axis system for positioning an X-ray imager, according to various embodiments.

FIG. 11 schematically illustrates a side view of a radiation therapy system with a four-bar linkage for positioning an X-ray imager, according to various embodiments.

FIG. 12 schematically illustrates a side view of a radiation therapy system with a radial-shaft-based system for positioning an X-ray imager, according to various embodiments.

FIG. 13 schematically illustrates a side view of a radiation therapy system with a longitudinal translation mechanism for positioning an X-ray imager, according to various embodiments.

FIG. 14 is a perspective view of a conical truss for a radiation therapy system, according to various embodiments.

FIG. 15 schematically illustrates a side view of the radiation therapy system of FIG. 14 with a conical truss incorporated into a rotatable gantry of the radiation therapy system, according to various embodiments.

16A schematically illustrates a plan view of a radiation therapy system, according to various embodiments.

FIG. 16B schematically illustrates a front view of the radiation therapy system of FIG. 16, according to various embodiments.

FIG. 17 schematically illustrates a side view of the radiation therapy system of FIGS. 16A and 16B with a movable collision shield, according to various embodiments.

FIG. 18 schematically illustrates a plan view of the radiation therapy system of FIGS. 16A and 16B and enclosed portions of a funnel-shaped cavity, according to various embodiments.

FIG. 19 schematically illustrates a front view of the radiation therapy system of FIGS. 16A and 16B and a bore opening, according to various embodiments.

FIG. 20 schematically illustrates a front view of a radiation therapy system with vertically actuated front shutters, according to various embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the disclosure, as generally described herein, and illustrated in the figures, can be arranged, substituted, combined, and designed in a wide variety of different configurations, all of which are explicitly contemplated and make part of this disclosure. Although the terms “first,” “second,” and “third” are used to describe various elements, these elements should not be limited by these terms. These terms are used to distinguish one element from another. For example, a first element may be referred to as a second element, and vice versa. Independent of the grammatical term usage, individuals with male, female or other gender identities are included within the term.

### INTRODUCTION

As noted previously, radiation therapy systems commonly employ drive systems that include either an O-ring linear accelerator (LINAC) or a C-arm LINAC. O-ring LINACs have the benefit of operating at higher rotational speeds and the advantage of being less susceptible to large deflections that can affect dosing accuracy. However, because O-ring LINACs require a patient to be positioned within a bore of the radiation therapy system, the possible orientations of the patient relative to the system are limited. By contrast, C-arm LINACs do not have a bore, and therefore the patient couch can be rotated relative to the system. As a result, more orientations of the patient relative to the plane of rotation of the LINAC are possible with a C-arm LINAC, which enables greater versatility of both treatment and imaging. However, because of the collision hazard associated with the open architecture of C-arm LINACs, the rotational speeds of C-arm LINACs are limited so that a minimum stop distance of the LINAC can be achieved in the event of a collision. Consequently, radiation therapies that benefit from higher rotational speeds of the LINAC sometimes cannot be implemented with a C-arm LINAC.

Accordingly, there is a need in the art for improved radiation therapy systems that have the versatility of a C-arm LINAC and the advantages of an O-ring LINAC.

### System Overview

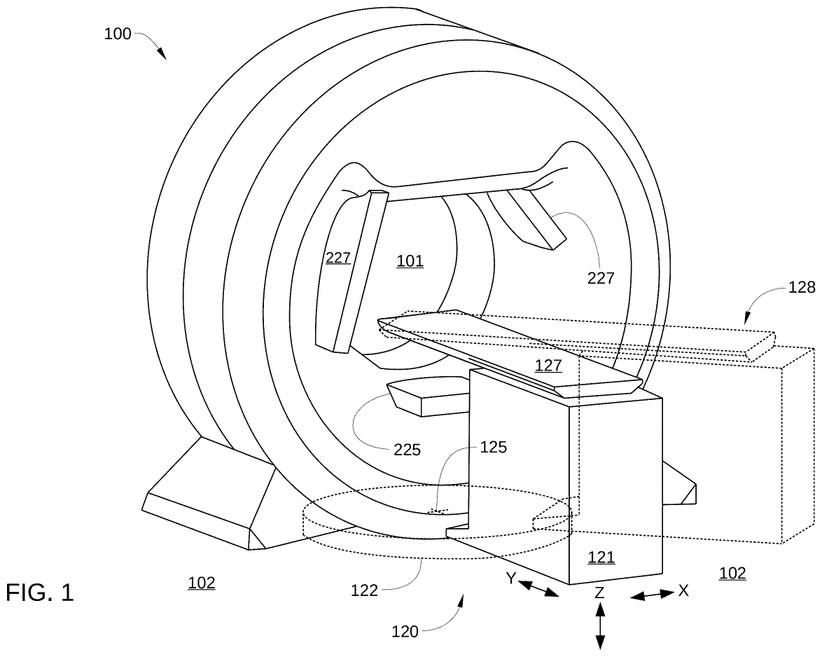

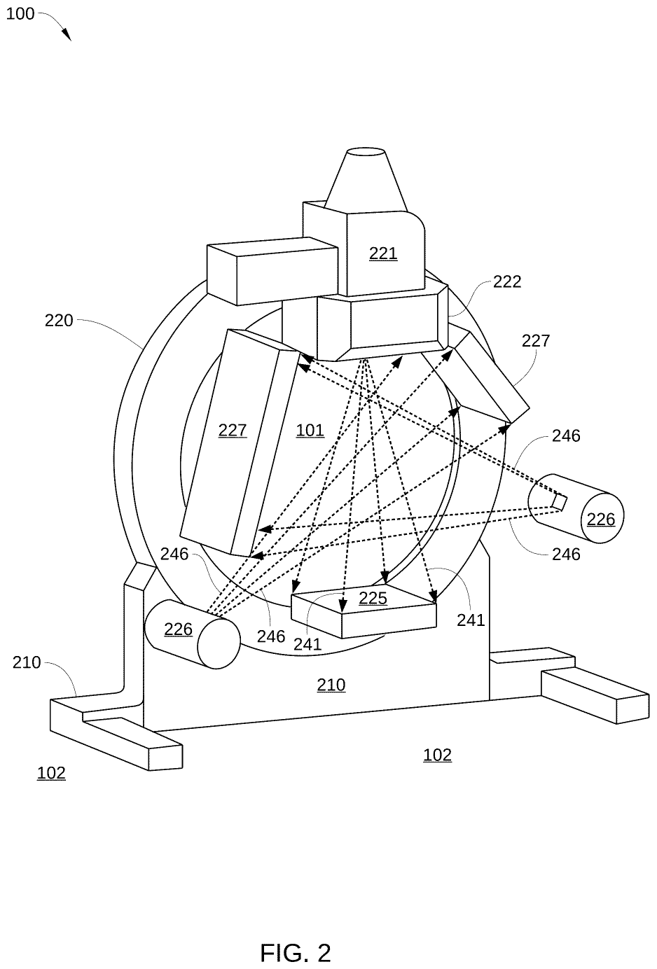

FIG. 1 is a perspective view of a radiation therapy (RT) system 100, according to various embodiments. FIG. 2 is a perspective view of RT system 100 with various covers and a couch-positioning system 120 omitted for clarity, according to various embodiments. RT system 100 is a radiation system that may be configured to detect intra-fraction motion in near real time using either optical or X-ray imaging techniques, or both. Thus, in some embodiments, RT system 100 is configured to provide stereotactic radiosurgery, image-guided radiation therapy (IGRT), intensity-modulated radiation therapy (IMRT), and/or other precision radiotherapy for lesions, tumors, and conditions anywhere in the body where radiation treatment is indicated. According to various embodiments, RT system 100 is capable of the fast gantry rotation of a fixed bore O-ring LINAC (for example when repositionable bore covers protect the patient and/or imaging components are repositionable) and the versatile couch positioning of a C-arm LINAC. In some embodiments, RT system 100 has a hybrid architecture that includes a rotatable gantry 220 and a rotatable couch 127 for positioning a patient (not shown) at different angles relative to the plane of rotation of rotatable gantry 220. Rotatable gantry 220 has one or more imaging systems and a treatment-delivering radiation source, such as a linear accelerator (LINAC) 221, mounted thereon, and therefore is configured to rotate the radiation source and the one or more imaging systems about a treatment bore 101 of RT system 100. As shown, treatment bore 101 is an open bore, rather than a cylinder that is closed on one end. Consequently, the potential for a patient positioned within treatment bore 101 to feel confined or constricted is reduced, which can greatly facilitate lengthier treatment sessions.

In the embodiment illustrated in FIGS. 1 and 2, RT system 100 includes one or more of LINAC 221, which generates an MV treatment beam 241 of high energy X-rays or other radiation, one or more imaging X-ray sources 226 (e.g., kilovolt (kV) x-ray sources), one or more X-ray imagers 227 (e.g., imaging panels), and a mega-Volt (MV) electronic portal imaging device (EPID) 225, all mounted on rotatable gantry 220. RT system 100 further includes treatment bore 101, a couch positioning system 120, and a couch 127 disposed on couch positioning system 120. In some embodiments, RT system 100 further includes one or more touchscreens, an image acquisition and treatment control computer, and couch motion controls (not shown), which are disposed within a treatment room with rotatable gantry 220, and a remote control console, which is disposed outside the treatment room and enables treatment delivery and patient monitoring from a remote location. In some embodiments, RT system 100 is capable of X-ray imaging of a target volume immediately prior to and/or during application of an MV treatment beam, so that an IGRT and/or an IMRT process can be guided by X-ray imaging. For example, in some embodiments, such processes can include kV imaging of the target volume in conjunction with imaging generated by MV treatment beam 241.

LINAC 221 can be a radiation source, and typically includes one or more of an electron gun for generating electrons, an accelerating waveguide, an electron beam target, an electron beam transport means (such as a bending magnet) for directing the electron beam to the electron beam target, and/or a collimator assembly 222 for collimating and shaping a treatment beam (such as MV treatment beam 241) that originates from the electron beam target. The collimator assembly 222 typically includes one or more of a primary collimator that defines the largest available circular radiation field the treatment beam, a secondary collimator for providing a rectangular or square radiation field at an isocenter of RT system 100 (for example via X-jaws and Y-jaws), and/or a multileaf collimator (MLC) for conforming the treatment beam to a planning target volume (PTV) or other anatomical target. In other embodiments, LINAC 221 can be any other radiation source suitable for radiation therapy.

Couch positioning system 120 is configured to precisely position couch 127 with respect to treatment bore 101, and includes a couch base 121 and a turntable 122. In some embodiments, couch positioning system 120 is mechanically coupled to a drive stand 210 (shown in FIG. 2) of RT system 100, and adjustably positions couch 127 relative to treatment bore 101 and LINAC 221. In some embodiments, couch base 121 is rotatably coupled to turntable 122, which is disposed under and coupled to drive stand 210. For example, in some embodiments, a pivot point and/or other components of turntable 122 are sunk into a support surface 102 that is external to RT system 100, such as a floor of a radiotherapy treatment facility.

In the embodiment illustrated in FIG. 1, couch positioning system 120 is configured to position couch 127 along lateral directions X, longitudinal directions Y, and vertical directions Z. Movement of couch 127 along lateral directions X corresponds to horizontal movement toward one side or the other side of treatment bore 101, movement along longitudinal directions Y corresponds to horizontal movement into or out of treatment bore 101, and movement along vertical directions Z corresponds to vertical movement toward or away from support surface 102. In addition, couch positioning system 120 can rotate couch 127 about a center of rotation 125, for example via turntable 122. Thus, couch positioning system 120 is rotatably coupled to support surface 102. In such embodiments, center of rotation 125 of turntable 122 is vertically aligned with a treatment isocenter of RT system 100 (not shown in FIGS. 1 and 2). As a result, when couch positioning system 120 rotates couch 127 about center of rotation 125, an anatomical region of a patient on couch 127 that is located at the treatment isocenter can be positioned with a different orientation relative to the treatment isocenter with little or no translation of the anatomical region relative to the treatment isocenter.

In FIG. 1, couch 127 is shown longitudinally extended from couch positioning system 120 into treatment bore 101 of RT system 100. For reference, couch positioning system 120 is also shown in an alternative rotated position 128 (dashed lines). The couch motion controls of RT system 100 can include input devices, such as buttons and/or switches, that enable a user to operate couch positioning system 120 to automatically and precisely position couch 120 to a predetermined location with respect to treatment bore 101. The couch motion controls also enable a user to manually position couch 127 to a particular location, such as a planned treatment position for a patient or anatomical target.

As shown schematically in FIG. 2, RT system 100 includes a drive stand 210 and rotatable gantry 220. Drive stand 210 is a stationary support structure for components of RT system 100, including rotatable gantry 220 and a drive system (not shown for clarity) for rotatably moving rotatable gantry 220. Drive stand 210 rests on and/or is fixed to support surface 102, which is external to treatment delivery system 200, such as a floor of a radiotherapy treatment facility. Rotatable gantry 220 is rotationally coupled to drive stand 210 via a rotatable coupling (such as a bearing) and is a support structure on which various components of treatment delivery system 200 are mounted, including LINAC 221, EPID 225, one or more imaging X-ray sources 226, and one or more X-ray imagers 227. During operation of treatment delivery system 200, rotatable gantry 220 rotates about treatment bore 101 when actuated by the drive system.

The drive system for rotatable gantry 220 rotationally actuates rotatable gantry 220 about treatment bore 101 and an isocenter of RT system 100. In some embodiments, the drive system includes a linear motor that can be fixed to drive stand 210 and interacts with a magnetic track (not shown) mounted on rotatable gantry 220. In other embodiments, the drive system includes another suitable drive mechanism for precisely rotating rotatable gantry 220 about treatment bore 101. LINAC 221 generates an MV treatment beam 241 of high energy X-rays (or in some embodiments electrons, protons, and/or other heavy charged particles, ultra-high dose rate X-rays (e.g., for FLASH radiotherapy) or microbeams for microbeam radiation therapy), and EPID 225 is configured to acquire X-ray images with MV treatment beam 241. Imaging X-ray sources 226 are configured to direct a conical beam of X-rays, referred to herein as imaging X-rays 246, through an isocenter of RT system 100 to a corresponding X-ray imager 227, where the isocenter typically corresponds to the location of a target volume (not shown) to be treated. In the embodiment illustrated in FIG. 2, X-ray imagers 227 and EPID 225 are depicted as a planar devices, whereas in other embodiments, X-ray imagers 227 and/or EPID 225 can have a curved configuration.

Each X-ray imager 227 receives imaging X-rays 246 from a corresponding X-ray source 226 and generates suitable projection images therefrom. According to certain embodiments, such projection images can then be employed to construct or update portions of imaging data for a digital volume that corresponds to a three-dimensional (3D) region that includes a target volume. That is, a 3D image of such a 3D region is reconstructed from the projection images. In some embodiments, cone-beam computed tomography (CBCT) and/or digital tomosynthesis (DTS) can be used to process the projection images generated by X-ray imager 227. CBCT is often employed at the beginning of a radiation therapy session to generate a set-up 3D reconstruction. For example, CBCT may be employed immediately prior to application of treatment beam 246 to generate a 3D reconstruction confirming that target volume has not moved or changed shape.

### Ring Gantry Architecture with Rotatable Couch

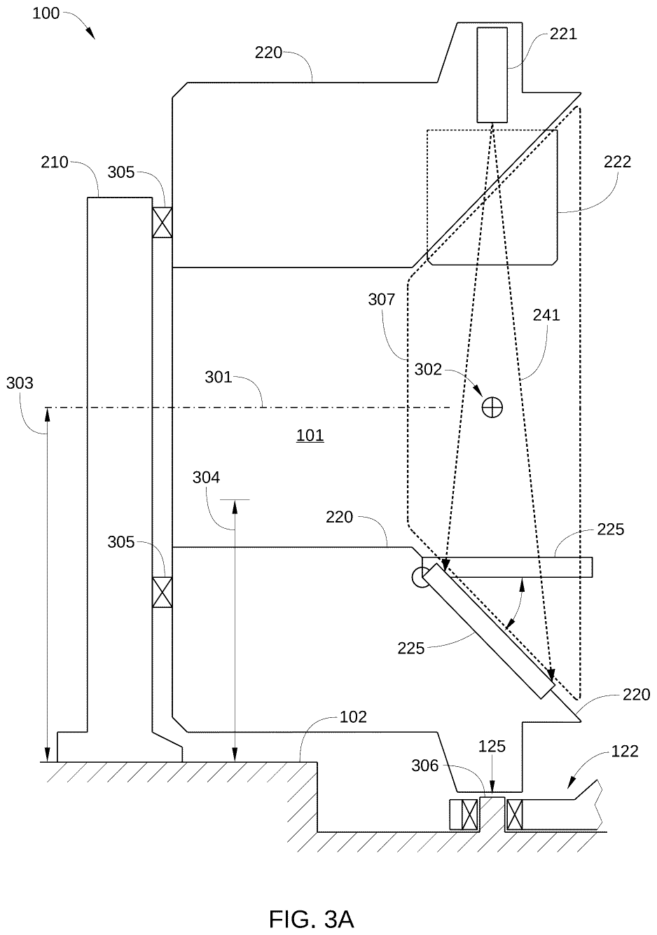

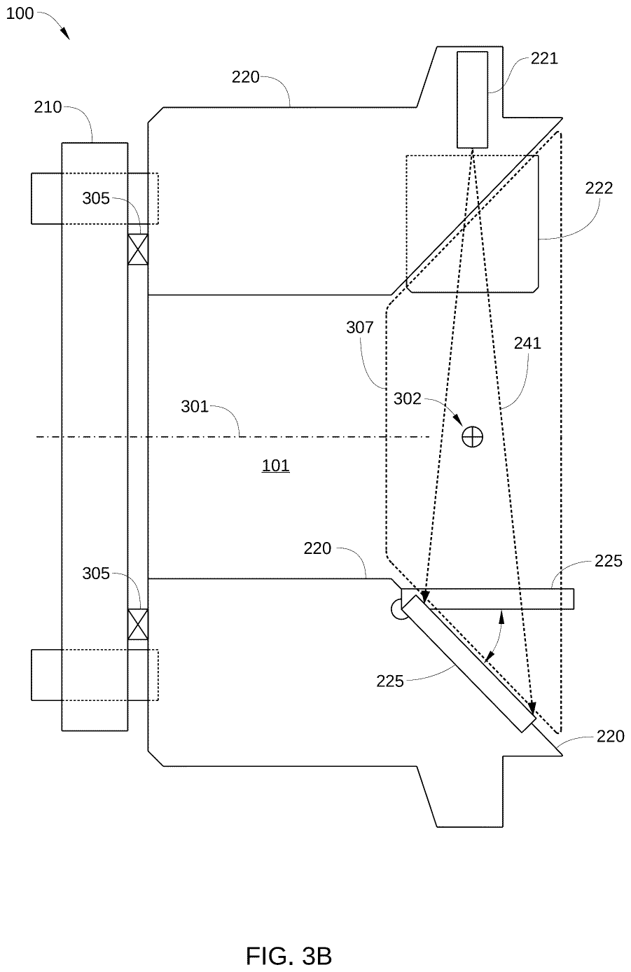

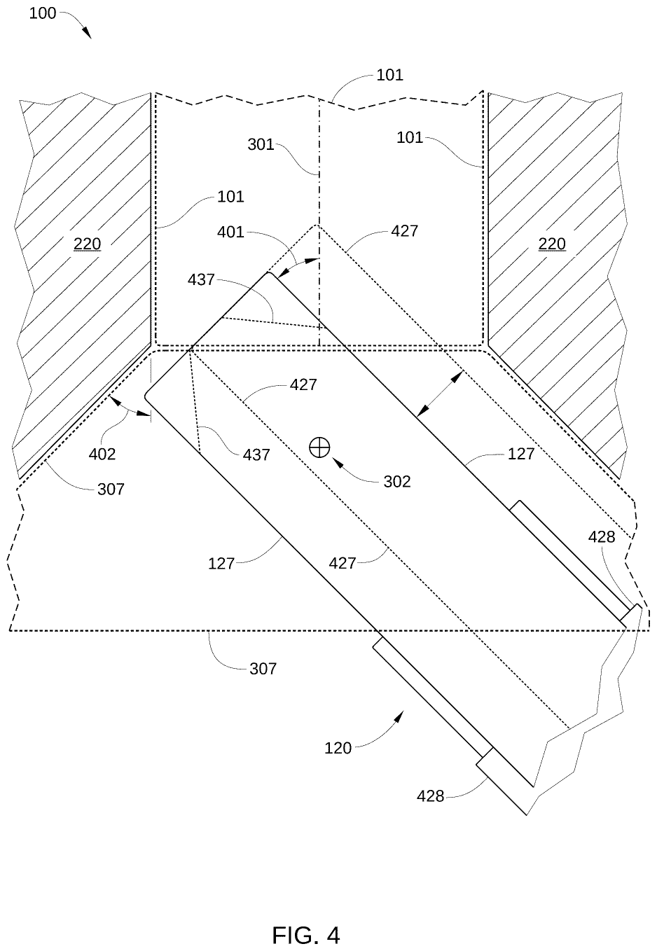

In some embodiments, a radiation therapy system includes an O-ring LINAC with a couch that is rotatable about an isocenter of the radiation therapy system. The radiation therapy system includes an open bore and a funnel-shaped cavity adjacent to the open bore that enables rotation of the couch relative to the isocenter without a collision threat for a patient on the couch. Examples of such embodiments are described below in conjunction with FIGS. 3A, 3B and 4.

FIG. 3A schematically illustrates a side view of RT system 100, according to various embodiments, and FIG. 3B schematically illustrates a plan view of RT system 100, according to various embodiments. For clarity, in FIGS. 3A and 3B, rotatable gantry 220 is shown in cross-section and couch 127 and couch base 121 are omitted. For purposes of description, in FIG. 3A rotatable gantry 220 is rotated so that LINAC 221 is vertically aligned with and positioned above an isocenter 302 of RT system 100, while in FIG. 3B rotatable gantry is rotated so that LINAC 221 is horizontally aligned with isocenter 302.

As shown, drive stand 210 supports rotatable gantry 220, which rotates about an isocenter 302 of RT system 100 and about a longitudinal axis 301 of treatment bore 101. Thus, an axis of rotation of rotatable gantry 220 is parallel and/or congruent with longitudinal axis 301 of treatment bore 101. Drive stand 210 is mounted on or otherwise coupled to support surface 102, and rotatable gantry 220 is rotationally coupled to drive stand 210, for example via a bearing 305. During operation of RT treatment system 100, LINAC 221 directs MV treatment beam 241 through isocenter 302 and toward EPID 225. In addition, one or both imaging X-ray sources 226 (not shown) direct imaging X-rays (not shown) through isocenter 302 and toward a corresponding X-ray imager 227. In the embodiment illustrated in FIG. 3A, turntable 122 is positioned relative to rotatable gantry 220 so that center of rotation 125 of pivot point 306 is vertically aligned with isocenter 302.

In the embodiment illustrated in FIG. 3A, turntable 122 is sunk into or installed below support surface 102 to provide additional clearance for rotatable gantry 220. Thus, in such embodiments, a height 303 of isocenter 302 above support surface 102 can be reduced. As a result, a height of a patient (not shown) treated by RT system 100 above support surface 102 is also reduced, thereby facilitating patient setup prior to treatment. In some embodiments, such a patient height can be further reduced when couch positioning system 120 operates with a loading height 304 for couch 127 that is below height 303 of isocenter 302 (couch positioning system 120 and couch 127 are shown in FIGS. 1 and 2). In such embodiments, couch positioning system 120 lowers couch 127 to loading height 304 for patient setup and raises couch 127 to a treatment height, at which an anatomical target volume is positioned at height 303 of isocenter 302. Example of such an anatomical target include a PTV, a gross tumor volume (GTV), a clinical target volume (CTV), and/or an internal target volume (ITV), among others.

In the embodiment illustrated in FIGS. 3A and 3B, RT system 100 includes a funnel-shaped cavity 307 (such as a truncated cone) that includes isocenter 302 and is adjacent to treatment bore 101. Funnel-shaped cavity 307 is a region that is free of rotating imaging components associated with rotatable gantry 220, while a component of LINAC 221, such as collimator assembly 222 may still extend into funnel-shaped cavity. Therefore, funnel-shaped cavity 307 is a region in which couch 127 and a patient disposed thereon can be located with a significantly reduced collision threat while rotatable gantry 220 rotates about isocenter 302 of RT system 100. In some embodiments, funnel-shaped cavity 307 is generally free of rotating imaging components, and in other embodiments, funnel-shape cavity 307 is formed when one or more imagers are retracted, rotated, or otherwise repositioned away from isocenter 302. For example, in the embodiment illustrated in FIGS. 3A and 3B, EPID 225 can be rotated away from isocenter 302 as shown to form funnel-shaped cavity 307. In such embodiments, other imaging components (not visible in FIGS. 3A and 3B) that are mounted on rotatable gantry 220 can be similarly rotated away from isocenter 302 to form funnel-shaped cavity 307. As a result, in such embodiments, couch positioning system 120 can rotate rotatable couch 127 via turntable 122 so that rotatable couch 127 is not longitudinally aligned with longitudinal axis 301 of treatment bore 101, but cannot collide with imaging components of rotatable gantry 220. Thus, in such embodiments, rotatable gantry 220 can rotate about longitudinal axis 301 during treatment with a significantly reduced collision threat to a patient on couch 127. For example, in such embodiments, rotatable gantry 220 can rotate in large arcs about longitudinal axis 301 without a collision threat to a patient on couch 127, even when couch 127 is rotated as much as about 45 degrees from alignment with longitudinal axis 301. One such embodiment is described below in conjunction with FIG. 4.

FIG. 4 schematically illustrates a plan view of funnel-shaped cavity 307 with couch 127 positioned therein, according to various embodiments. As shown, funnel-shaped cavity 307 is disposed adjacent to treatment bore 101, and couch 127 has been rotated about isocenter 302 by a rotation angle 401 relative to longitudinal axis 301 of treatment bore 101 and isocenter 302 of RT system 100. Funnel-shaped cavity 307 enables couch 127 to be rotated by rotation angle 401 as shown without a collision threat from imaging components of rotatable gantry 220 (cross-hatched) when rotatable gantry 220 rotates about longitudinal axis 301 of treatment bore 101 over a large and clinically significant gantry rotation arc (e.g., ½ to ¾ of a rotation). Similarly, a patient disposed on couch 127 can also be rotated relative to isocenter 302 by rotation angle 401 as shown without a collision threat from imaging components of rotatable gantry 220. Consequently, RT system 100 can perform radiation therapy treatments that generally are restricted to C-arm LINAC systems and cannot be implemented with a conventional O-ring LINAC. Further, because a patient disposed on couch 127 is located within funnel-shaped cavity 307, and therefore is free of a collision threat from imaging components of rotatable gantry 220, radiation treatments that benefit from higher rotational speeds of rotatable gantry 220 can be performed by RT system 100. By contrast, C-arm LINAC systems that allow the rotation of a treatment couch with respect to an isocenter as shown in FIG. 4 are generally limited in the rotational velocity of the C-arm gantry so that a minimum stop distance of the C-arm gantry can be achieved in the event of a collision, for example by exposed components of rotatable gantry 220 such as deployed imaging components and/or a collimator assembly. Thus, in some instances, RT system 100 can perform radiation treatments that cannot be performed by either conventional slow-moving C-arm LINACs or conventional less-flexible O-ring LINACs.

In some embodiments, funnel-shaped cavity 307 is based at least in part on a cone angle 402 of a surface of rotatable gantry 220. For example, in some embodiments, cone angle 402 of a surface of rotatable gantry 220 is selected to allow rotation of couch 127 about isocenter 302 so that rotation angle 401 equals a specific target angle value (e.g., 40°, 45°, 50°, etc.) relative to longitudinal axis 301 of treatment bore 101. In the embodiment illustrated in FIG. 4, cone angle 402 is further based at least in part on enabling funnel-shaped cavity 307 to accommodate couch 127 being positioned at a maximum lateral position 427 (indicated by dashed lines in FIG. 4) when couch 127 is rotated so that rotation angle 401 equals the target angle value. In some embodiments, maximum lateral position 427 of couch 127 corresponds to couch 127 being positioned at a maximum lateral travel on a couch pedestal 428 that can be implemented by couch positioning system 120. Thus, in such embodiments, funnel-shaped cavity 307 is configured so that a patient can be disposed on couch 127 and be free of collision threat by imaging components of rotating gantry 220 when couch 127 is positioned at maximum lateral position 427 and rotation angle 401 equals the target angle value.

In the embodiment illustrated in FIG. 4, couch 127 has a rectangular footprint. In other embodiments, couch 127 has a reduced footprint to reduce a collision threat by exposed components of rotatable gantry 220, such as deployed imaging components and/or a collimator assembly. In such embodiments, couch 127 can have a footprint or perimeter that is smaller than the rectangular footprint shown in FIG. 4. For example, in some embodiments, couch 127 has a triangular or trapezoidal footprint 437 (dashed lines), or a shape approximating a silhouette of a head of a patient.

### Ring Gantry Architecture with Angled X-Ray Imager(s)

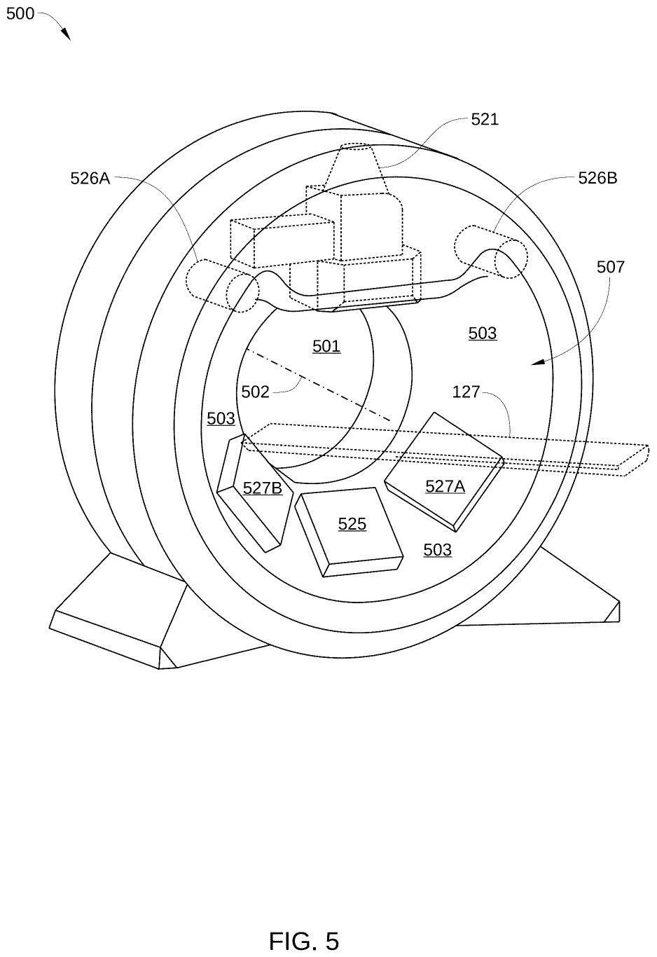

In some embodiments, funnel-shaped cavity 307 of RT system 100 is formed in part by one or more X-ray imagers that are coupled to rotatable gantry 220. In such embodiments, each of the one or more imagers has an X-ray-receiving surface that is oriented at a non-orthogonal angle to X-rays received by that X-ray imager, such as X-ray that pass through an isocenter of the radiation therapy system. For example, in some embodiments, the one or more X-ray imagers are coupled to rotatable gantry 220 at a non-orthogonal angle to rotatable gantry 220. In such embodiments, the one or more X-ray imagers do not extend into funnel-shaped cavity 307, and therefore are not a collision threat to a patient on couch 127 when couch 127 is rotated to rotation angle 401 and rotatable gantry 220 rotates. Examples of such embodiments are described below in conjunction with FIGS. 5-8.

FIG. 5 is a perspective view of an RT system 500 with angled X-ray imagers, according to various embodiments. In the embodiment illustrated in FIG. 5, the angled X-ray imagers include a first X-ray imager 527A, a second X-ray imager 527B, and an EPID 525, all mounted on or coupled to a rotatable gantry. The rotatable gantry is not visible in FIG. 5, but in some embodiments can be consistent with rotatable gantry 220 in FIG. 2. Also shown in FIG. 5 are a first imaging X-ray source 526A, a second imaging X-ray source 526B, and a LINAC 521 with a collimator assembly 522 (dashed lines), which are also mounted on or coupled to the rotatable gantry. In the embodiment illustrated in FIG. 5, first X-ray imager 527A, second X-ray imager 527B, and EPID 525 are depicted as a planar devices, whereas in other embodiments, first X-ray imager 527A, second X-ray imager 527B, and/or EPID 525 can have a curved configuration.

In some embodiments, first X-ray imager 527A, second X-ray imager 527B, and EPID 525 do not extend past or are substantially flush with an isocenter-facing surface 503 of the rotatable gantry of RT system 500. Thus, in such embodiments, isocenter-facing surface 503 and the X-ray-receiving surfaces of first X-ray imager 527A, second X-ray imager 527B, and EPID 525 define a funnel-shaped cavity 507. As a result, when couch 127 (dashed lines) is rotated as shown to a rotation angle within funnel-shaped cavity 507 relative to a longitudinal axis 502 of treatment bore 501 of RT system 500, first X-ray imager 527A, second X-ray imager 527B, and EPID 525 do not create a collision threat to couch 127 or to a patient disposed on couch 127.

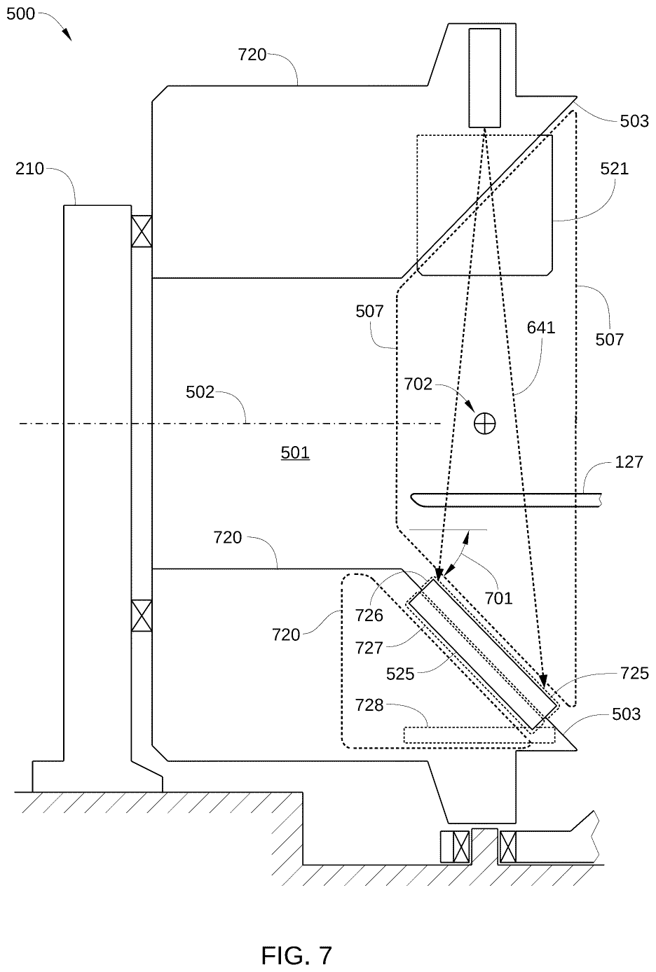

In the embodiment illustrated in FIG. 5, first X-ray imager 527A, second X-ray imager 527B, and EPID 525 are not positioned with an X-ray-receiving surface that is flush with isocenter-facing surface 503 of the rotatable gantry. Instead, first X-ray imager 527A, second X-ray imager 527B, and EPID 525 are positioned so that a surface portion of each imager extends past isocenter-facing surface 503 towards an isocenter (not shown) of RT system 500. The extension of EPID 525 past isocenter-facing surface 503 is shown more clearly in FIG. 7. Thus, in the embodiment illustrated in FIG. 5, portions of funnel-shaped cavity 507 are formed by the X-ray receiving surfaces of first X-ray imager 527A, second X-ray imager 527B, and EPID 525. Alternatively, in other embodiments, first X-ray imager 527A, second X-ray imager 527B, and EPID 525 are positioned with an X-ray-receiving surface that is flush with isocenter-facing surface 503 of the rotatable gantry. In either case, first X-ray imager 527A, second X-ray imager 527B, and EPID 525 are positioned relative to longitudinal axis 502 so that rotation of the rotatable gantry does not cause a collision threat to couch 127 or a patient disposed on couch 127.

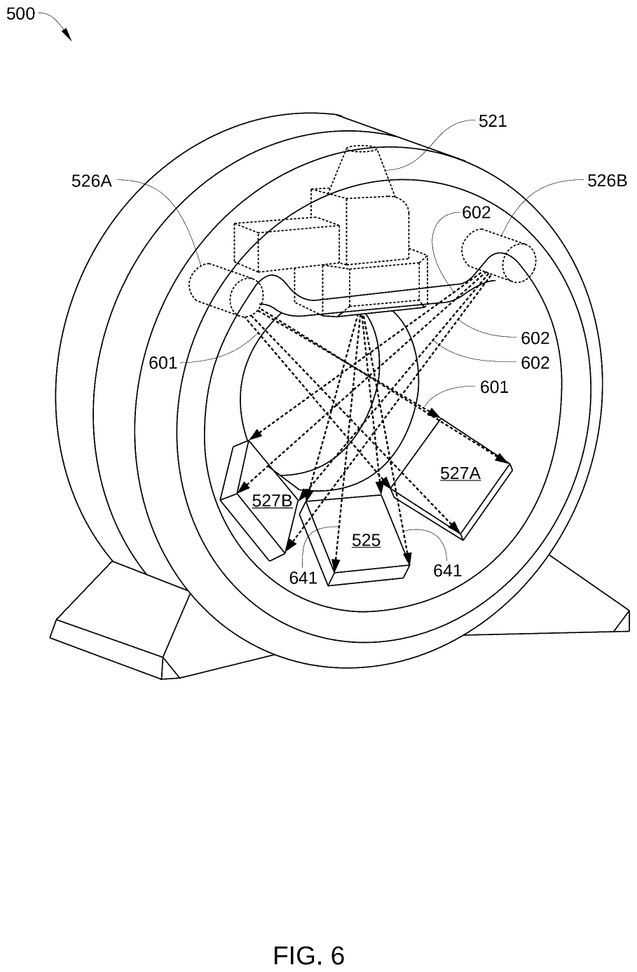

FIG. 6 is a perspective view of RT system 500 with X-ray imagers receiving X-rays, according to various embodiments. In the embodiment illustrated in FIG. 6, first X-ray imager 527A receives imaging X-rays 601 from first imaging X-ray source 526A, second X-ray imager 527B receives imaging X-ray 602 from second imaging X-ray source 526B, and EPID 525 receives an MV treatment beam 641 from LINAC 521. As shown in FIG. 6, first X-ray imager 527A, second X-ray imager 527B, and EPID 525 receive X-rays while oriented at a non-orthogonal angle to the X-rays being received. Thus, in such embodiments, each of first X-ray imager 527A, second X-ray imager 527B, and EPID 525 has an X-ray-receiving surface that is oriented at a non-orthogonal angle to X-rays received by that X-ray imager. One such embodiment is described in greater detail below in conjunction with FIG. 7.

FIG. 7 schematically illustrates a side view of RT system 500, according to various embodiments. For clarity, in FIG. 7, a rotatable gantry 720 of RT system 500 is shown in cross-section. In some embodiments, rotatable gantry 720 of RT system 500 can be consistent with rotatable gantry 220 of RT system 100 (shown in FIG. 2). For reference, a portion of couch 127 is shown disposed within funnel-shaped cavity 507. For purposes of description, in FIG. 7 rotatable gantry 720 is rotated so that LINAC 521 is vertically aligned with and positioned above an isocenter 702 of RT system 500.

As shown, drive stand 210 supports rotatable gantry 720, which rotates about isocenter 702 of RT system 500 and about longitudinal axis 502 of treatment bore 501. In the embodiment illustrated in FIG. 7, EPID 525 is oriented at a cone angle 701 of funnel-shaped cavity 507 relative to longitudinal axis 502 of treatment bore 501. Thus, in such embodiments, a cross-section of EPID 525 is oriented parallel to isocenter-facing surface 503 of rotatable gantry 720. As a result, EPID 525 has an x-ray-receiving surface 725 that is oriented at a non-orthogonal angle to X-rays received by EPID 525, such as the portion of MV treatment beam 641 that passes through isocenter 702. Additionally or alternatively, in some embodiments, some or all other X-ray imagers mounted to rotatable gantry 720 are similarly oriented relative to longitudinal axis 502. Thus, in such embodiments, each of EPID 525, first X-ray imager 527A, and/or second X-ray imager 527B of RT system 500 has an X-ray-receiving surface that is oriented at a non-orthogonal angle to X-rays being received, and each X-ray-receiving surface can form a portion of the boundary of funnel-shaped cavity 507. In such embodiments, imaging performed by EPID 525 generally includes one or more modified reconstruction algorithms that enable accurate imaging to be performed when X-ray-receiving surface 725 is oriented at a non-orthogonal angle to X-rays received by EPID 525.

In the embodiment illustrated in FIG. 7, an X-ray-receiving surface 725 of EPID 525 is not flush with isocenter-facing surface 503 of rotatable gantry 720. Instead, in the embodiment shown, X-ray receiving surface 725 extends past isocenter-facing surface 503 toward isocenter 702. Thus, in such embodiments, EPID 525 includes a front portion 726 and a rear portion 727, where front portion 726 receives MV treatment beam 641 and extends past isocenter-facing surface 503 toward isocenter 702 and rear portion 727 extends from isocenter-facing surface 503 away from isocenter 702. As shown, EPID 525 is fixed in position outside of funnel-shaped cavity 507 and oriented at cone angle 701 of funnel-shaped cavity 507. As a result, in such embodiments, a region 721 within rotatable gantry 720 can be employed to mount or contain other components or systems associated with RT system 500 that can be beneficially mounted on or within rotatable gantry 720. Also shown in FIG. 7 is an alternative configuration of EPID 525 in which EPID 525 is fixed in a position 728 (dashed lines) outside of funnel-shaped cavity 507 and is not oriented at cone angle 701 of funnel-shaped cavity 507. Instead, in position 728, X-ray-receiving surface 725 of EPID 525 is oriented at an orthogonal angle to X-rays received by EPID 525, such as the portion of MV treatment beam 641 that passes through isocenter 702. In such embodiments, region 721 within rotatable gantry 720 is generally not available, but conventional imaging algorithms can be employed with EPID 525. Further, in such embodiments, EPID 525 is subject to less scatter during imaging.

In some embodiments, LINAC 521 is of sufficient size that LINAC 521 and/or components associated with LINAC 521, such as collimator assembly 522, extends into funnel-shaped cavity 507. As a result, LINAC 521, collimator assembly 522, and/or the like can potentially collide with a couch 127 or a patient (not shown) disposed on couch 127 when couch 127 is rotated to a rotation angle similar to rotation angle 401 in FIG. 4. In such embodiments, RT system 500 can be configured to include software controls and/or mechanical constraints for rotatable gantry 720 that prevent rotation of LINAC 521 and/or collimator assembly 522 into an area that can be occupied by a patient or couch 127. As a result, in embodiments in which LINAC 521 and/or collimator assembly 522 extends into funnel-shaped cavity 507, LINAC 521 and/or collimator assembly 522 is not a significant collision threat to the patient or to couch 127. For example, in instances of head treatments using RT system 500, the potential for a collision with collimator assembly 522 can be reduced or eliminated due to the smaller size of the head relative to body. Specifically, MV treatment beam 641 from LINAC 521 can reach a target volume within a head of a patient when LINAC 521 and collimator assembly 522 are positioned on a far side of the head of the patient, and does not need to originate from a position near a shoulder of the patient. Thus, LINAC 521 and collimator assembly 522 can be positioned on a side of couch 127 that is not rotated close to rotatable gantry 720. For example, in FIG. 4, rotatable gantry 720 can be rotated so that LINAC 521 and/or collimator assembly 522 is disposed on the left side of FIG. 4 while couch 127 is rotated toward the right side of FIG. 4. As a result, LINAC 521 and/or collimator assembly 522 cannot collide with couch 127 and/or a patient on couch 127 unless rotated over to the right side of FIG. 4. It is noted that LINAC 521 and/or collimator assembly 522 can be rotated up to about ¾ of a rotation about couch 127 without the risk of being rotated to the right side of FIG. 4 and potentially colliding with couch 127 and/or a patient on couch 127. Treatment methods requiring imaging for target supervision during treatment with couch 127 rotated can be implemented over a ¾ rotation arc. Thus, RT system 500 can implement such treatment methods even with couch 127 rotated as shown in FIG. 4, which is generally not possible with either conventional C-arm LINACs or conventional O-ring LINACs.

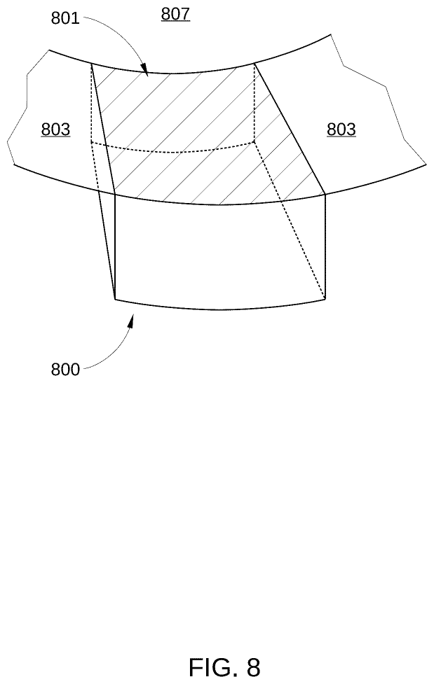

In some embodiments, an X-ray imager of RT system 500 that is coupled to rotatable gantry 720 and oriented at cone angle 701 of funnel-shaped cavity 507 can have a curved configuration. In such embodiments, an X-ray-receiving surface of the X-ray imager can facilitate a more expansive funnel-shaped cavity 507 that is free of collision threats. One such embodiment is described below in conjunction with FIG. 8.

FIG. 8 is a schematic perspective view of an X-ray imager 800 of a radiotherapy system that has a curved configuration, according to various embodiments. In the embodiment illustrated in FIG. 8, X-ray imager 800 is shown relative to an isocenter-facing surface 803 of a rotatable gantry (not shown), which can be consistent with isocenter-facing surface 503 of rotatable gantry 720 in FIG. 7. In some embodiments, X-ray imager 800 can be implemented as an X-ray imager that is coupled to the rotatable gantry and is oriented at a cone angle of a funnel-shaped cavity 807 that is formed in part by isocenter-facing surface 803. In some embodiments, funnel-shaped cavity 807 can be consistent with funnel-shaped cavity 507 in FIGS. 5-7. Thus, in some embodiments, X-ray imager 800 can be implemented as one or more of first X-ray imager 527A, second X-ray imager 527B, and/or EPID 525.

As shown, X-ray imager 800 includes an X-ray-receiving surface 801 (cross-hatched) that is curved and not planar. In some embodiments, X-ray-receiving surface 801 is curved to match a curvature of isocenter-facing surface 803. In such embodiments, X-ray imager 800 can be positioned as shown relative to isocenter-facing surface 803, so that X-ray-receiving surface 801 is flush with isocenter-facing surface 803. In such embodiments, X-ray-receiving surface 801 forms a portion of a boundary of funnel-shape cavity 807. In other embodiments, X-ray imager 800 can extend past isocenter-facing surface 803 toward an isocenter (not shown) of the radiotherapy system.

### Ring Gantry Architecture with Repositionable X-Ray Imager(s)

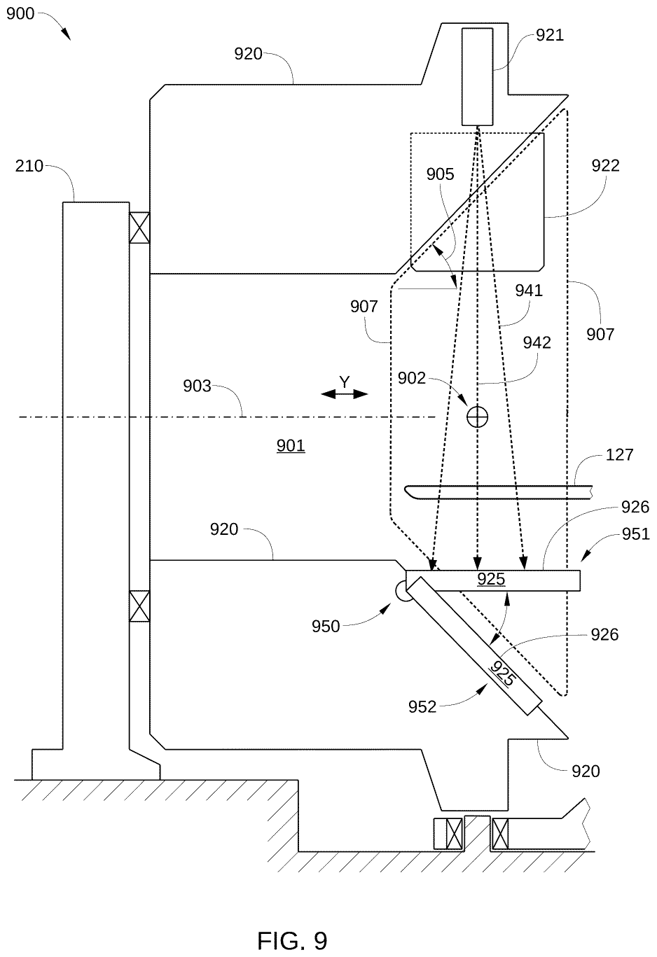

In some embodiments, a funnel-shaped cavity of a radiation therapy system, such as funnel-shaped cavity 307 of FIG. 3, is formed when one or more X-ray imagers are selectively positioned from a first imaging position to a second imaging position. In such embodiments, when in the first imaging position, each of the one or more imagers has an X-ray-receiving surface that is oriented to be orthogonal to X-rays that pass through an isocenter of the radiation therapy system and are received by the X-ray imager. By contrast, when in the second imaging position, each of the one or more X-ray imagers has an X-ray-receiving surface that is oriented to be non-orthogonal to X-rays that pass through the isocenter and are received by the X-ray imager. According to various embodiments, different mechanisms can be employed to selectively position the one or more X-ray imagers between a first imaging position and a second imaging position. Examples of such embodiments are described below in conjunction with FIGS. 9-13.

FIG. 9 schematically illustrates a side view of an RT system 900 with a rotational mechanism 950 for positioning an X-ray imager, according to various embodiments. For clarity, in FIG. 9, a rotatable gantry 920 of RT system 900 is shown in cross-section. For reference, a portion of couch 127 is shown disposed within a funnel-shaped cavity 907 of RT system 900. For purposes of description, in FIG. 9 rotatable gantry 920 is rotated so that a LINAC 921 and collimator assembly 922 is vertically aligned with and positioned above an isocenter 902 of RT system 900. In some embodiments, rotatable gantry 920 of RT system 900 can be consistent with rotatable gantry 220 of RT system 100 (shown in FIG. 2). Further, in some embodiments, RT system 900 can be consistent with RT system 100 of FIGS. 1-3, except that one or more X-ray imagers 925 of RT system 900 are coupled to rotatable gantry 920 by rotational mechanism 950, which selectively positions the one or more X-ray imagers 925 between a first imaging position 951 and a second imaging position 952 with respect to isocenter 902.

In the embodiment illustrated in FIG. 9, X-ray imager 925 is depicted as an EPID positioned on rotatable gantry 920 to receive an MV treatment beam 941 from LINAC 921, for example after passing through isocenter 902. Additionally or alternatively, in some embodiments, X-ray imager 925 can be implemented as an X-ray imager consistent with X-ray imagers 227 in FIGS. 1 and 2. As shown, drive stand 210 supports rotatable gantry 920, which rotates about isocenter 902 of RT system 900 and about a longitudinal axis 903 of a treatment bore 901. In the embodiment illustrated in FIG. 9, rotational mechanism 950 can be a rotational mechanism that enables the selective positioning of X-ray imager 925 between first imaging position 951 and second imaging position 952, such as a hinge, bearing, or the like.

In some embodiments, when X-ray imager 925 is in first imaging position 951, an X-ray-receiving surface 926 is oriented orthogonal to X-rays 942 that pass through isocenter 902 and are received by X-ray imager 925. In some embodiments, when X-ray imager 925 is in second imaging position 952, X-ray-receiving surface 926 is oriented at a non-orthogonal angle to X-rays 942. In the embodiment illustrated in FIG. 9, when X-ray imager 925 is in second imaging position 952, X-ray imager 925 is oriented at a cone angle 905 of funnel-shaped cavity 907. In such embodiments, the non-orthogonal angle of X-ray-receiving surface 926 is cone angle 905. In other embodiments, when X-ray imager 925 is in second imaging position 952, rotational mechanism 950 can orient X-ray imager 925 to any other suitable angle that prevents X-ray imager 925 from being a collision hazard within funnel-shaped cavity 907.

As shown, when X-ray imager 925 is in first imaging position 951, most or all of X-ray imager 925 is disposed within funnel-shaped cavity 907. Thus, when couch 127 is rotated about isocenter 902 to an alternate rotated position (such as alternative rotated position 128 shown in FIG. 1) and X-ray imager 925 is in first imaging position 951, X-ray imager 925 can be a collision hazard to couch 127 and/or a patient disposed on couch 127. However, when couch 127 is aligned in longitudinal directions Y with treatment bore 901, X-ray imager 925 can be in first imaging position 951 without being a collision hazard while rotatable gantry 920 rotates about treatment bore 901. This is because, when couch 127 is aligned in longitudinal directions Y with treatment bore 901, RT system 900 is configured as a conventional O-ring LINAC, and components of rotatable gantry 920 disposed within funnel-shaped cavity 907 are not collision hazards. By contrast, when X-ray imager 925 is in second imaging position 952, X-ray imager 925 is disposed outside of funnel-shaped cavity 907. Therefore, couch 127 can be rotated about isocenter 902 to an alternate rotated position and X-ray imager 925 is not a collision hazard to couch 127 and/or a patient disposed on couch 127. Consequently, when X-ray imager 925 is in second imaging position 952, RT system 900 can be configured to operate as a C-arm LINAC with a rotated couch with no collision hazards with imaging components. It is noted that, in some embodiments, imaging performed by X-ray imager 925 when in second imaging position 952 generally includes one or more modified reconstruction algorithms that enable accurate imaging to be performed when X-ray-receiving surface 926 is oriented at a non-orthogonal angle to X-rays 942.

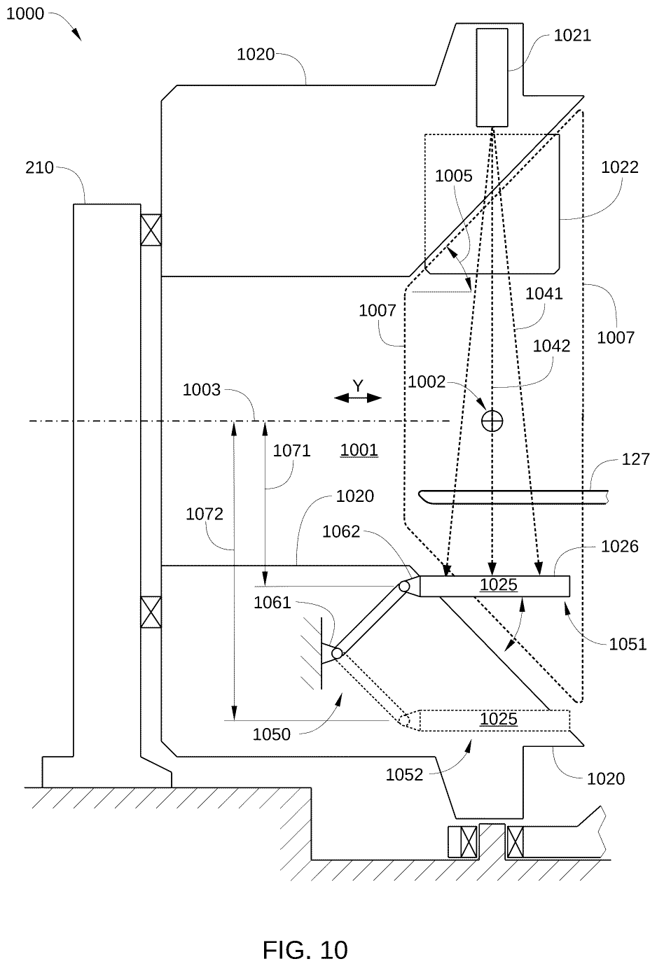

FIG. 10 schematically illustrates a side view of an RT system 1000 with a dual rotary-axis system 1050 for positioning an X-ray imager, according to various embodiments. For clarity, in FIG. 10, a rotatable gantry 1020 of RT system 1000 is shown in cross-section. For reference, couch 127 is shown disposed within a funnel-shaped cavity 1007 of RT system 1000. For purposes of description, in FIG. 10 rotatable gantry 1020 is rotated so that a LINAC 1021 and/or a collimator assembly 1022 is vertically aligned with and positioned above an isocenter 1002 of RT system 1000. In some embodiments, rotatable gantry 1020 of RT system 1000 can be consistent with rotatable gantry 220 of RT system 100 (shown in FIG. 2). Further, in some embodiments, RT system 1000 can be consistent with RT system 100 of FIGS. 1-3, except that one or more X-ray imagers 1025 of RT system 1000 are coupled to rotatable gantry 1020 by dual rotary-axis system 1050, which selectively positions the one or more X-ray imagers 1025 between a first imaging position 1051 and second imaging position 1052 (dashed lines) with respect to isocenter 1002.

In the embodiment illustrated in FIG. 10, X-ray imager 1025 is depicted as an EPID positioned on rotatable gantry 1020 to receive an MV treatment beam 1041 from LINAC 1021, for example after passing through isocenter 1002. Additionally or alternatively, in some embodiments, X-ray imager 1025 can be implemented as an X-ray imager consistent with X-ray imagers 227 in FIGS. 1 and 2. As shown, drive stand 210 supports rotatable gantry 1020, which rotates about isocenter 1002 of RT system 1000 and about a longitudinal axis 1003 of a treatment bore 1001.

In the embodiment illustrated in FIG. 10, dual rotary-axis system 1050 can be a mechanism that enables the selective positioning of X-ray imager 1025 between first imaging position 1051 and a second imaging position 1052 via a first rotary axis 1061 and a second rotary axis 1062. In such embodiments, first rotary axis 1061 is coupled to rotary gantry 1020 and second rotary axis 1062 is coupled to X-ray imager 1025 as shown. In operation, dual rotary-axis system 1050 selectively translates or otherwise moves X-ray imager 1025 between first imaging position 1051 and second imaging position 1052. In the embodiment illustrated in FIG. 10, dual rotary-axis system 1050 radially translates (relative to longitudinal axis 1003) X-ray imager 1025 between first imaging position 1051 and second imaging position 1052. Consequently, when X-ray imager 1025 is in either first imaging position 1051 or second imaging position 1052, an X-ray-receiving surface 1026 of X-ray imager 1025 is oriented orthogonal to X-rays 1042 that pass through isocenter 1002 and are received by X-ray imager 1025 and can acquire X-ray images.

When X-ray imager 1025 is in first imaging position 1051, X-ray imager 1025 is disposed at a first radial distance 1071 from isocenter 1002. When X-ray imager 1025 is in second imaging position 1052, X-ray imager 1025 is disposed at a second radial distance 1072 from isocenter 1002. As shown in FIG. 10, second radial distance 1072 is greater than first radial distance 1071.

As shown, when X-ray imager 1025 is in first imaging position 1051, most or all of X-ray imager 1025 is disposed within funnel-shaped cavity 1007. Thus, when couch 127 is rotated about isocenter 1002 to an alternate rotated position (such as alternative rotated position 128 shown in FIG. 1) and X-ray imager 1025 is in first imaging position 1051, X-ray imager 1025 can be a collision hazard to couch 127 and/or a patient disposed on couch 127. However, when couch 127 is aligned in longitudinal directions Y with treatment bore 1001, X-ray imager 1025 can be in first imaging position 1051 without being a collision hazard while rotatable gantry 1020 rotates about treatment bore 1001. Conversely, when X-ray imager 1025 is in second imaging position 1052, X-ray imager 1025 is disposed outside of funnel-shaped cavity 1007. Therefore, couch 127 can be rotated about isocenter 1002 to an alternate rotated position and X-ray imager 1025 is not a collision hazard to couch 127 and/or a patient disposed on couch 127.

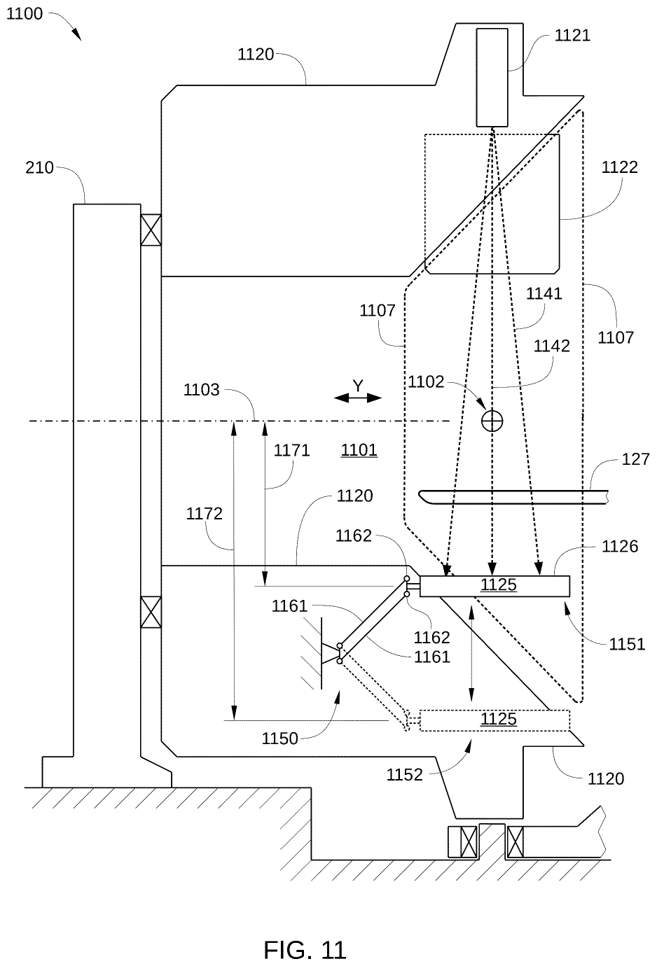

FIG. 11 schematically illustrates a side view of an RT system 1100 with a four-bar linkage 1150 for positioning an X-ray imager, according to various embodiments. For clarity, in FIG. 11, a rotatable gantry 1120 of RT system 1100 is shown in cross-section. For reference, couch 127 is shown disposed within a funnel-shaped cavity 1107 of RT system 1100. For purposes of description, in FIG. 11 rotatable gantry 1120 is rotated so that a LINAC 1121 and/or collimator assembly 1122 is vertically aligned with and positioned above an isocenter 1102 of RT system 1100. In some embodiments, rotatable gantry 1120 of RT system 1100 can be consistent with rotatable gantry 220 of RT system 110 (shown in FIG. 2). Further, in some embodiments, RT system 1100 can be consistent with RT system 100 of FIGS. 1-3, except that one or more X-ray imagers 1125 of RT system 1100 are coupled to rotatable gantry 1120 by four-bar linkage 1150, which selectively positions the one or more X-ray imagers 1125 between a first imaging position 1151 and a second imaging position 1152 (dashed lines) with respect to isocenter 1102.

In the embodiment illustrated in FIG. 11, X-ray imager 1125 is depicted as an EPID positioned on rotatable gantry 1120 to receive an MV treatment beam 1141 from LINAC 1121, for example after passing through isocenter 1102. Additionally or alternatively, in some embodiments, X-ray imager 1125 can be implemented as an X-ray imager consistent with X-ray imagers 227 in FIGS. 1 and 2. As shown, drive stand 210 supports rotatable gantry 1120, which rotates about isocenter 1102 of RT system 1100 and about a longitudinal axis 1103 of a treatment bore 1101.

In the embodiment illustrated in FIG. 11, four-bar linkage 1150 can be a mechanism that enables the selective positioning of X-ray imager 1125 between first imaging position 1151 and second imaging position 1152 via a four links 1161 joined by four joints 1162. As shown, four-bar linkage 1150 movably couples X-ray imager 1125 to rotatable gantry 1120, and in operation selectively translates or otherwise moves X-ray imager 1125 between first imaging position 1151 and second imaging position 1152. In the embodiment illustrated in FIG. 11, four-bar linkage 1150 radially translates (relative to longitudinal axis 1103) X-ray imager 1125 between first imaging position 1151 and second imaging position 1152. Consequently, when X-ray imager 1125 is in either first imaging position 1151 or second imaging position 1152, an X-ray-receiving surface 1126 of X-ray imager 1125 is oriented orthogonal to X-rays 1142 that pass through isocenter 1102 and are received by X-ray imager 1125 and can acquire X-ray images.

When X-ray imager 1125 is in first imaging position 1151, X-ray imager 1125 is disposed at a first radial distance 1171 from isocenter 1102. When X-ray imager 1125 is in second imaging position 1152, X-ray imager 1125 is disposed at a second radial distance 1172 from isocenter 1102. As shown in FIG. 11, second radial distance 1172 is greater than first radial distance 1171.

As shown, when X-ray imager 1125 is in first imaging position 1151, most or all of X-ray imager 1125 is disposed within funnel-shaped cavity 1107. Thus, when couch 127 is rotated about isocenter 1102 to an alternate rotated position (such as alternative rotated position 128 shown in FIG. 1) and X-ray imager 1125 is in first imaging position 1151, X-ray imager 1125 can be a collision hazard to couch 127 and/or a patient disposed on couch 127. However, when couch 127 is aligned in longitudinal directions Y with treatment bore 1101, X-ray imager 1125 can be in first imaging position 1151 without being a collision hazard while rotatable gantry 1120 rotates about treatment bore 1101. Conversely, when X-ray imager 1125 is in second imaging position 1152, X-ray imager 1125 is disposed outside of funnel-shaped cavity 1107. Therefore, couch 127 can be rotated about isocenter 1102 to an alternate rotated position and X-ray imager 1125 is not a collision hazard to couch 127 and/or a patient disposed on couch 127.

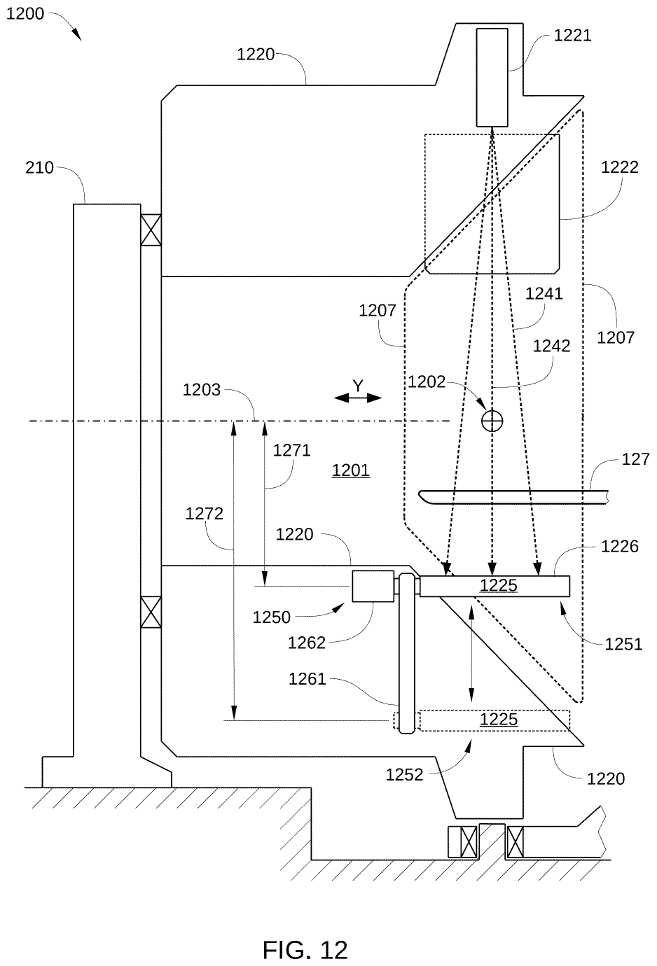

FIG. 12 schematically illustrates a side view of an RT system 1200 with a radial-shaft-based system 1250 for positioning an X-ray imager, according to various embodiments. For clarity, in FIG. 12, a rotatable gantry 1220 of RT system 1200 is shown in cross-section. For reference, couch 127 is shown disposed within a funnel-shaped cavity 1207 of RT system 1200. For purposes of description, in FIG. 12 rotatable gantry 1220 is rotated so that a LINAC 1221 and/or collimator assembly 1222 is vertically aligned with and positioned above an isocenter 1202 of RT system 1200. In some embodiments, rotatable gantry 1220 of RT system 1200 can be consistent with rotatable gantry 220 of RT system 120 (shown in FIG. 2). Further, in some embodiments, RT system 1200 can be consistent with RT system 100 of FIGS. 1-3, except that one or more X-ray imagers 1225 of RT system 1200 are coupled to rotatable gantry 1220 by radial-shaft-based system 1250, which selectively positions the one or more X-ray imagers 1225 between a first imaging position 1251 and a second imaging position 1252 (dashed lines) with respect to isocenter 1202.

In the embodiment illustrated in FIG. 12, X-ray imager 1225 is depicted as an EPID positioned on rotatable gantry 1220 to receive an MV treatment beam 1241 from LINAC 1221, for example after passing through isocenter 1202. Additionally or alternatively, in some embodiments, X-ray imager 1225 can be implemented as an X-ray imager consistent with X-ray imagers 227 in FIGS. 1 and 2. As shown, drive stand 210 supports rotatable gantry 1220, which rotates about isocenter 1202 of RT system 1200 and about a longitudinal axis 1203 of a treatment bore 1201.

In the embodiment illustrated in FIG. 12, radial-shaft-based system 1250 can include a mechanism that enables the selective positioning of X-ray imager 1225 between first imaging position 1251 and second imaging position 1252 via a radial shaft 1261 and an associated actuator 1262. In some embodiments, actuator 1262 of radial-shaft-based system 1250 includes one of a spindle drive (e.g., an electric motor that drives a threaded spindle), a pneumatic drive, a servo drive, or any other suitable actuation system for selectively positioning X-ray imager 1125 between first imaging position 1251 and second imaging position 1252 via radial shaft 1261.

As shown, radial-shaft-based system 1250 movably couples X-ray imager 1225 to rotatable gantry 1220, and in operation selectively translates or otherwise moves X-ray imager 1225 between first imaging position 1251 and second imaging position 1252. In the embodiment illustrated in FIG. 12, radial-shaft-based system 1250 radially translates (relative to longitudinal axis 1203) X-ray imager 1225 between first imaging position 1251 and second imaging position 1252. Consequently, when X-ray imager 1225 is in either first imaging position 1251 or second imaging position 1252, an X-ray-receiving surface 1226 of X-ray imager 1225 is oriented orthogonal to X-rays 1242 that pass through isocenter 1202 and are received by X-ray imager 1225 and can acquire X-ray images.

When X-ray imager 1225 is in first imaging position 1251, X-ray imager 1225 is disposed at a first radial distance 1271 from isocenter 1202. When X-ray imager 1225 is in second imaging position 1252, X-ray imager 1225 is disposed at a second radial distance 1272 from isocenter 1202. As shown in FIG. 12, second radial distance 1272 is greater than first radial distance 1271.

As shown, when X-ray imager 1225 is in first imaging position 1251, most or all of X-ray imager 1225 is disposed within funnel-shaped cavity 1207. Thus, when couch 127 is rotated about isocenter 1202 to an alternate rotated position (such as alternative rotated position 128 shown in FIG. 1) and X-ray imager 1225 is in first imaging position 1251, X-ray imager 1225 can be a collision hazard to couch 127 and/or a patient disposed on couch 127. However, when couch 127 is aligned in longitudinal directions Y with treatment bore 1201, X-ray imager 1225 can be in first imaging position 1251 without being a collision hazard while rotatable gantry 1220 rotates about treatment bore 1201. Conversely, when X-ray imager 1225 is in second imaging position 1252, X-ray imager 1225 is disposed outside of funnel-shaped cavity 1207. Therefore, couch 127 can be rotated about isocenter 1202 to an alternate rotated position and X-ray imager 1225 is not a collision hazard to couch 127 and/or a patient disposed on couch 127.

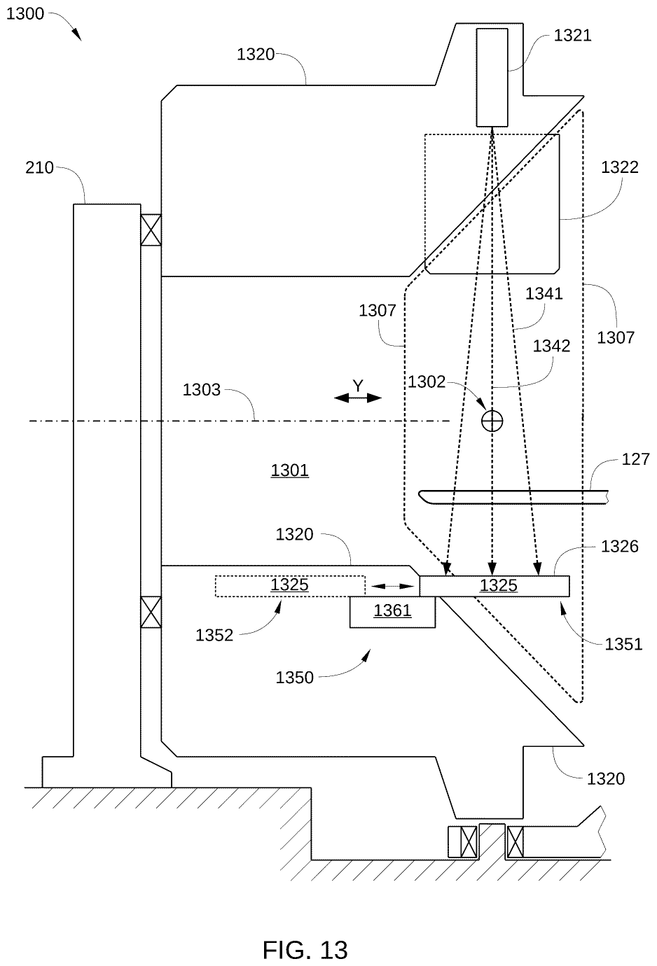

FIG. 13 schematically illustrates a side view of an RT system 1300 with a longitudinal translation mechanism 1350 for positioning an X-ray imager, according to various embodiments. For clarity, in FIG. 13, a rotatable gantry 1320 of RT system 1300 is shown in cross-section. For reference, couch 127 is shown disposed within a funnel-shaped cavity 1307 of RT system 1300. For purposes of description, in FIG. 13 rotatable gantry 1320 is rotated so that a LINAC 1321 and/or collimator assembly 1322 is vertically aligned with and positioned above an isocenter 1302 of RT system 1300. In some embodiments, rotatable gantry 1320 of RT system 1300 can be consistent with rotatable gantry 220 of RT system 100 (shown in FIG. 2). Further, in some embodiments, RT system 1300 can be consistent with RT system 100 of FIGS. 1-3, except that one or more X-ray imagers 1325 of RT system 1300 are coupled to rotatable gantry 1320 by longitudinal translation mechanism 1350, which selectively positions the one or more X-ray imagers 1325 between a first imaging position 1351 and a second imaging position 1352 (dashed lines) with respect to isocenter 1302.

In the embodiment illustrated in FIG. 13, X-ray imager 1325 is depicted as an EPID positioned on rotatable gantry 1320 to receive an MV treatment beam 1341 from LINAC 1321, for example after passing through isocenter 1302. Additionally or alternatively, in some embodiments, X-ray imager 1325 can be implemented as an X-ray imager consistent with X-ray imagers 227 in FIGS. 1 and 2. As shown, drive stand 210 supports rotatable gantry 1320, which rotates about isocenter 1302 of RT system 1300 and about a longitudinal axis 1303 of a treatment bore 1301.

In the embodiment illustrated in FIG. 13, longitudinal translation mechanism 1350 can be a mechanism that enables the selective positioning of X-ray imager 1325 between first imaging position 1351 and second imaging position 1352 via a longitudinal (Y-direction) translation. For example, in some embodiments, longitudinal translation mechanism 1350 includes an associated actuator 1361. In some embodiments, actuator 1361 of longitudinal translation mechanism 1350 includes one of a spindle drive, a pneumatic drive, a servo drive, or any other suitable actuation system for selectively positioning X-ray imager 1125 between first imaging position 1351 and second imaging position 1352 longitudinally.

As shown, longitudinal translation mechanism 1350 movably couples X-ray imager 1325 to rotatable gantry 1320, and in operation selectively translates or otherwise moves X-ray imager 1325 between first imaging position 1351 and second imaging position 1352. In the embodiment illustrated in FIG. 13, longitudinal translation mechanism 1350 longitudinally translates (parallel to longitudinal axis 1303) X-ray imager 1325 between first imaging position 1351 and second imaging position 1352.

As shown, when X-ray imager 1325 is in first imaging position 1351, most or all of X-ray imager 1325 is disposed within funnel-shaped cavity 1307. Thus, when couch 127 is rotated about isocenter 1302 to an alternate rotated position (such as alternative rotated position 128 shown in FIG. 1) and X-ray imager 1325 is in first imaging position 1351, X-ray imager 1325 can be a collision hazard to couch 127 and/or a patient disposed on couch 127. However, when couch 127 is aligned in longitudinal directions Y with treatment bore 1301, X-ray imager 1325 can be in first imaging position 1351 without being a collision hazard while rotatable gantry 1320 rotates about treatment bore 1301. Conversely, when X-ray imager 1325 is in second imaging position 1352, X-ray imager 1325 is disposed outside of funnel-shaped cavity 1307. Therefore, couch 127 can be rotated about isocenter 1302 to an alternate rotated position and X-ray imager 1325 is not a collision hazard to couch 127 and/or a patient disposed on couch 127.

### Ring Gantry Architecture with Conical Truss

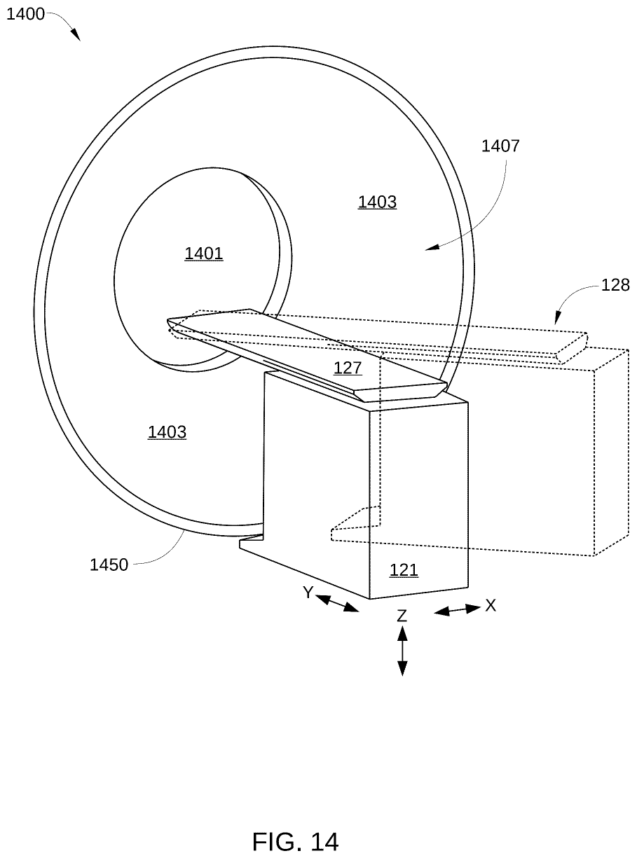

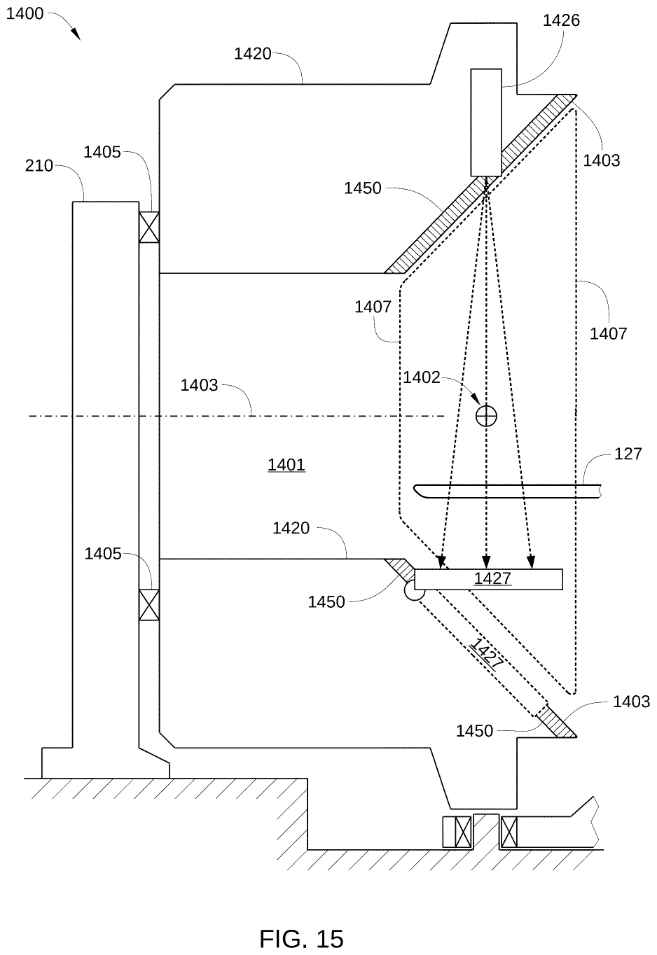

In some embodiments, a funnel-shaped cavity of a radiation therapy system is formed at least in part by an isocenter-facing surface of a rotatable gantry of the radiation therapy system. For example, in FIG. 5, isocenter-facing surface 503 of RT system 500 forms a portion of the boundary of funnel-shaped cavity 507. According to various embodiments, the isocenter-facing surface of the radiation therapy system can be a surface of a conical truss that supports one or more components mounted on rotatable gantry of the radiation therapy system. One such embodiment is described below in conjunction with FIGS. 14 and 15.

FIG. 14 is a perspective view of a conical truss 1450 for an RT system 1400, according to various embodiments. FIG. 15 schematically illustrates a side view of RT system 1400 with conical truss 1440 incorporated into a rotatable gantry 1420 of RT system 1400, according to various embodiments. For reference, couch 127 and couch base 121 are shown disposed within a funnel-shaped cavity 1407 of RT system 1400 that is formed in part by an isocenter-facing surface 1403 of conical truss 1450 (cross-hatched in FIG. 15). In some embodiments, an imaging X-ray source 1426, an X-ray imager 1427, a LINAC and/or collimator assembly, and an EPID are mounted on conical truss 1450. For clarity, in FIG. 14 imaging X-ray source 1426, X-ray imager 1427, the LINAC, collimator assembly, and the EPID are omitted.

As shown, conical truss 1450 is included in rotatable gantry 1420 of RT system 1400 and supports X-ray source 1426 and X-ray imager 1427, among other components of RT system 400. In some embodiments, conical truss 1450 has a centerline that is congruent with a longitudinal axis 1403 of a treatment bore 1401 of RT system 1400 and rotates about longitudinal axis 1403 when rotatable gantry 1420 rotates. In such embodiments, isocenter-facing surface 1403 of conical truss 1450 can form a portion of the boundary of funnel-shaped cavity 1407 of RT system 1400. Further, in some embodiments, conical truss 1450 supports imaging X-ray source 1426, X-ray imager 1427, the LINAC and/or collimator assembly of RT system 1400, and the EPID of RT system 1400. Due to the inherent rigidity of a conical form factor, coupling high-mass and/or precisely positioned components to rotatable gantry 1420 via conical truss 1450 can result in RT system 1400 being subject to significantly smaller deflections than in a conventional C-arm LINAC. Examples of such components include imaging X-ray source 1426, X-ray imager 1427, the LINAC and/or collimator assembly of RT system 1400, and the EPID of RT system 1400. In a conventional O-ring LINAC, such precisely positioned components are generally supported by a cantilever that is coupled to, for example, a back plate attached to a bearing 1405. As is well-known, deflection of a cantilever is a function of the square of the length of the cantilever. Therefore, when components supported by a cantilever have high mass and operate based on precise positioning, the cantilever can be subject to deflection that significantly affects the precision of the positioning of the components. By contrast, conical truss 1450 operates as a closed section when supporting components of rotatable gantry 1420. As a result, the deflection-causing loads associated with high-mass components coupled to rotatable gantry 1420 are distributed around the funnel shape of conical truss 1450, thereby enhancing precise and repeatable positioning of an isocenter 1402. An additional benefit of conical truss 1450 is that X-ray sources and collimators for imaging X-ray source 1426 can be disposed within rotatable gantry 1420 and behind conical truss 1450.

In some embodiments, conical truss 1450 has a truncated cone shape, as shown in FIGS. 14 and 15. Additionally or alternatively, in some embodiments, conical truss 1450 is implemented as or includes a truncated sheet metal cone. In such embodiments, conical truss 1450 can be formed from a single piece of sheet metal or from multiple pieces of sheet metal coupled together into a single assembly.

### Ring Gantry Architecture with Repositionable Bore Covers

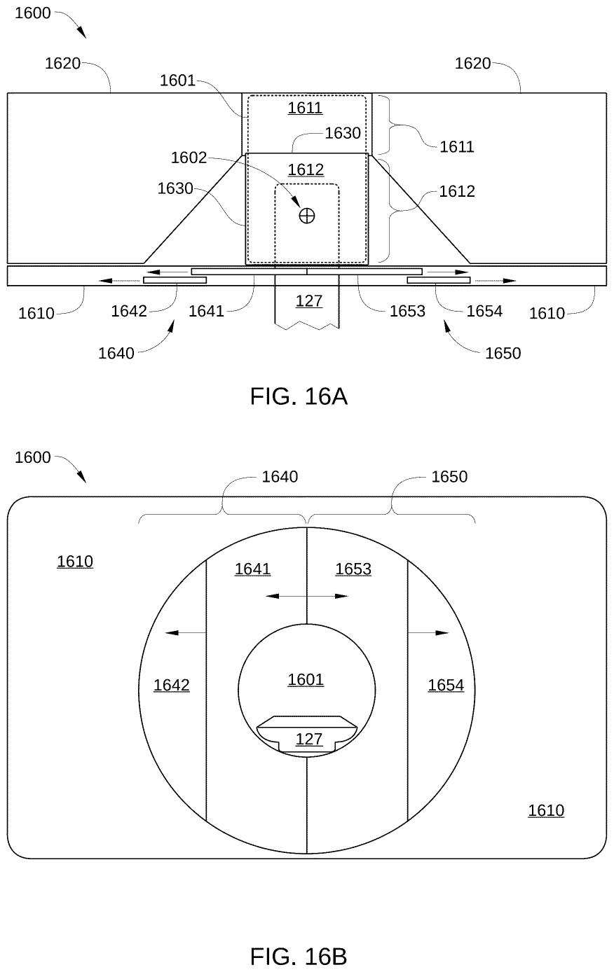

In some embodiments, a radiation therapy system includes one or more movable collision shields that can be selectively deployed to protect a patient in different treatment positions. For example, the one or more movable collision shields can be deployed to a first configuration when the patient is positioned on a couch that is aligned with and disposed within a bore of the radiation therapy system. Conversely, the one or more movable collision shields can be deployed to a second configuration when the patient is positioned on a couch that is rotated out of alignment with the bore, for example about an isocenter of the radiation therapy system. The movable collision shields can include an extendable bore cover and/or retractable front covers. The extendable bore cover can be deployed to extend collision protection into a funnel-shaped cavity between a fixed bore and the rotatable couch, while the retractable front covers can form a front facade of the radiation therapy system for protection of an operator from rotating components within the funnel-shaped cavity. Examples of such embodiments are described below in conjunction with FIGS. 16A-20.

FIG. 16A schematically illustrates a plan view of an RT system 1600, according to various embodiments, and FIG. 16B schematically illustrates a front view of RT system 1600, according to various embodiments. Also shown is couch 127 positioned in a bore 1601 of RT system 1600 and proximate an isocenter 1602 of RT system 1600. In the embodiment illustrated in FIGS. 16A and 16B, RT system 1600 includes a front cover 1610 and a rotatable gantry 1620, and can be consistent with RT system 100 of FIGS. 1-3. RT system further includes a movable collision shield 1630 and a first front shutter 1640 and a second front shutter 1650.

In the embodiment illustrated in FIGS. 16A and 16B, bore 1601 includes a fixed portion 1611 and an extendable portion 1612. Fixed portion 1611 of bore 1600 remains in place, while extendable portion 1612 is formed when movable collision shield 1630 is disposed at a first position within a funnel-shaped cavity of RT system 1600 as shown in FIG. 16A.

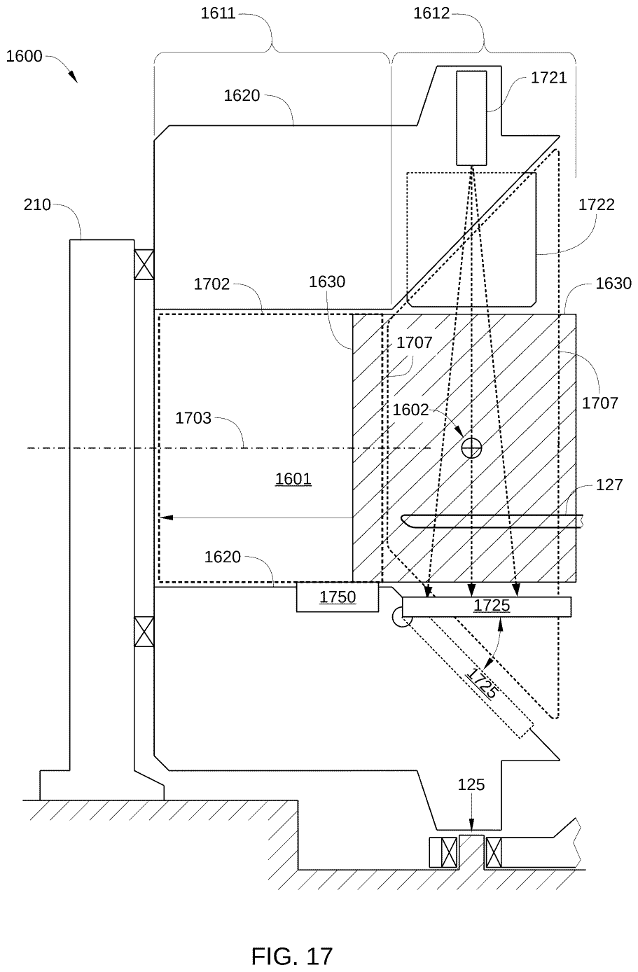

Movable collision shield 1630 is disposed between bore 1601 and rotatable gantry 1620, and is coupled to a positioning mechanism (not shown) that selectively positions movable collision shield 1630 between a first position and a second position. One embodiment of movable collision shield 1630 and the positioning mechanism is described below in conjunction with FIG. 17.

FIG. 17 schematically illustrates a side view of RT system 1600 with movable collision shield 1630, according to various embodiments. For clarity, in FIG. 17, rotatable gantry 1620 of RT system 1600 is shown in cross-section. For reference, couch 127 is shown disposed within a funnel-shaped cavity 1707 of RT system 1700. For purposes of description, in FIG. 17 rotatable gantry 1620 is rotated so that a LINAC 1721 and/or collimator assembly 1722 is vertically aligned with and positioned above isocenter 1602 of RT system 1600. In some embodiments, rotatable gantry 1620 of RT system 1600 can be consistent with rotatable gantry 220 of RT system 100 (shown in FIG. 2). Further, in some embodiments, RT system 1600 can be consistent with RT system 100 of FIGS. 1-3, except that RT system 1600 includes movable collision shield 1630 (cross-hatched).

As shown, movable collision shield 1630 is disposed in bore 1601, and is movably coupled to a positioning mechanism 1750. In the embodiment illustrated in FIG. 17, movable collision shield 1630 is an open-ended cylinder, and positioning mechanism 1750 translates movable collision shield 1630 between a first position (which is shown if FIG. 17) and a second position 1702 (dashed lines) in a direction parallel to a longitudinal axis 1703 of bore 1601. Positioning mechanism can include any suitable actuation system for selectively positioning movable collision shield 1630 between the first position and second position 1702. In the embodiment illustrated in FIG. 17, when movable collision shield 1630 is disposed at second position 1702, movable collision shield 1630 does not extend outside of bore 1601 towards drive stand 210. In other embodiments, movable collision shield 1630 can extend partially out of bore 1601 toward drive stand 210, for example when fixed portion 1611 of bore 1601 has a smaller longitudinal length than movable collision shield 1630.

As shown, when movable collision shield 1630 is disposed at the first position, at least a portion of movable collision shield 1630 is disposed within funnel-shaped cavity 1707 of RT system 1600. In some embodiments, in the first position, movable collision shield 1630 forms extendable portion 1612 of bore 1601, by shielding a patient (not shown) disposed on couch 127 from collision hazards when rotatable gantry 1620 rotates about bore 1601. For example, because movable collision shield 1630 is positioned between a patient disposed in an extendable portion 1612 of bore 1601, an X-ray imager 1725 that is positioned within funnel-shaped cavity 1707 is not a collision threat to the patient when rotatable gantry 1620 rotates. Thus, X-ray imager 1725 can be positioned as shown within funnel-shaped cavity 1701 when movable collision shield 1630 is positioned at the first position as shown.

It is noted that when movable collision shield 1630 is positioned at the first position, couch 127 generally cannot be rotated about center of rotation 125 due to interference between couch 127 and movable collision shield 1630. Thus, in some embodiments, for radiation therapy that includes rotation of couch 127 about center of rotation 125, movable collision shield 1630 is positioned at second position 1702. Because movable collision shield 1630 does not shield a patient on couch 127 from collision threats disposed within funnel-shaped cavity 1707, X-ray imager 1725 and/or other X-ray imagers are generally repositioned outside of funnel-shaped cavity 1707 when movable collision shield 1630 is disposed at second position 1702. In some embodiments, when movable collision shield 1630 is disposed at second position 1702, most or all of movable collision shield 1630 is disposed within fixed portion 1611 of bore 1601.

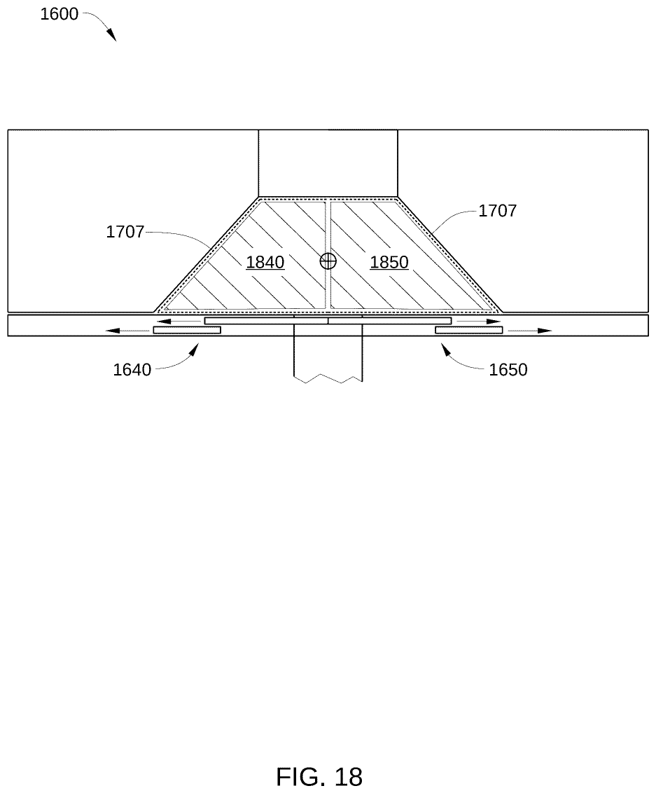

Returning to FIGS. 16A and 16B, first front shutter 1640 and second front shutter 1650 are retractable front covers for RT system 1600. In operation, first front shutter 1640 and second front shutter 1650 can form a front facade of RT system 1600 for protection of an operator from rotating components within a funnel-shaped cavity of RT system 1600, such as funnel-shaped cavity 1707 shown in FIG. 17. As such, in some embodiments, first front shutter 1640 is operable to selectively move between a first (closed) position (as shown in FIGS. 16A and 16B) and a second (open) position. Similarly, second front shutter 1650 is operable to selectively move between a third (closed) position (as shown in FIGS. 16A and 16B) and a fourth (open) position. Thus, when first front shutter 1640 is disposed in the first position, first front shutter encloses a first portion of a funnel-shaped cavity of RT system 1600, and when second front shutter 1650 is disposed in the third position, second front shutter 1650 encloses a second portion of a funnel-shaped cavity of RT system 1600. One embodiment of the first and second portions of the funnel-shaped cavity enclosed by first front shutter 1640 and second front shutter 1650 is described below in conjunction with FIG. 18.

FIG. 18 schematically illustrates a plan view of RT system 1600 and enclosed portions of funnel-shaped cavity 1707, according to various embodiments. As shown, first front shutter 1640 is disposed in a first (closed) position, and therefore first front shutter 1640 encloses a first portion 1840 (cross-hatched) of funnel-shaped cavity 1707 (dashed lines) of RT system 1600. Conversely, when first front shutter 1640 is disposed in the second (open) position, first front shutter 1640 exposes first portion 1840 of funnel-shaped cavity 1707. In a similar vein, second front shutter 1650 is disposed in a third (closed) position, and therefore second front shutter 1650 encloses a second portion 1850 (cross-hatched) of funnel-shaped cavity 1707. Conversely, when second front shutter 1650 is disposed in the fourth (open) position, second front shutter 1650 exposes second portion 1850 of funnel-shaped cavity 1707. To more clearly show funnel-shaped cavity 1707, movable collision shield 1630 of RT system 1600 is omitted in FIG. 18.

In the embodiment illustrated in FIG. 18, first portion 1840 is a left-hand portion of funnel-shaped cavity 1707 and second portion 1850 is a right-hand portion of funnel-shaped cavity 1707. In embodiments in which first front shutter 1640 and second front shutter 1650 open and close vertically, first portion 1840 can be an upper portion of funnel-shaped cavity 1707 and second portion 1850 can be a lower portion of funnel-shaped cavity 1707, or vice-versa.

Returning to FIGS. 16A and 16B, in some embodiments, first front shutter 1640 can be a single panel that is selectively moved between the first position and the second position or have multiple stages. Similarly, in some embodiments, second front shutter 1650 can be a single panel that is selectively moved between the first position and the second position or have multiple stages. In some instances, such a single-panel configuration can result in each panel having a width that extends beyond an outer width of front cover 1610 when in the open position. Consequently, in some embodiments, first front shutter 1640 and second front shutter 1659 each comprise multiple overlapping stages. For example, in the embodiment illustrated in FIGS. 16A and 16B, first front shutter 1640 includes a first stage 1641 and a second stage 1642, and second front shutter 1650 includes a third stage 1653 and a fourth stage 1654. Thus, in such embodiments, first front shutter 1640 and second front shutter 1659 each comprise two stages. In other embodiments, first front shutter 1640 and/or second front shutter 1659 each comprise three or more stages.

As shown, when first front shutter 1640 is disposed in the first (closed) position, first stage 1641 is deployed away from second stage 1642, for example toward second front shutter 1650. Similarly, when second front shutter 1650 is disposed in the third (closed) position, third stage 1653 is deployed away from fourth stage 1654, for example toward first front shutter 1640.

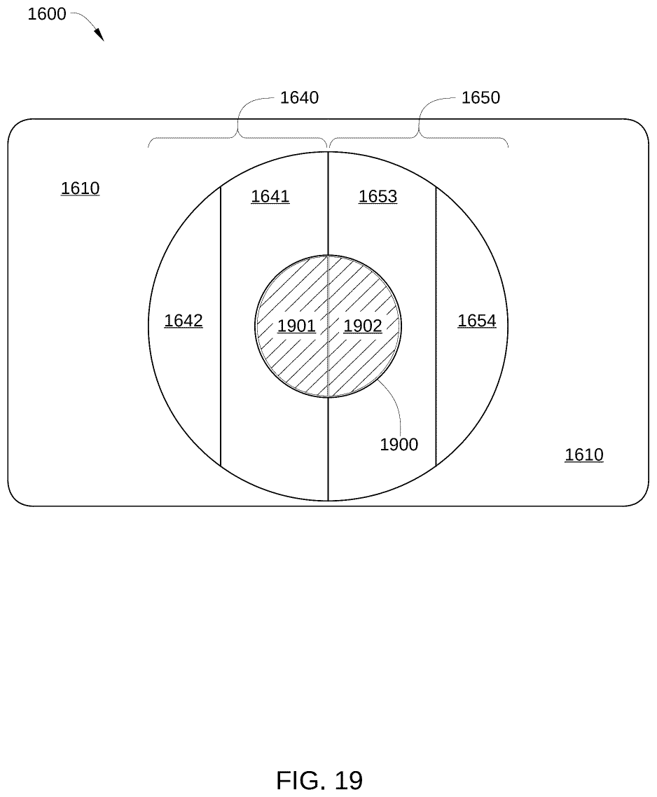

To enable access to bore 1601 when first front shutter 1640 and second front shutter 1650 are closed, in some embodiments, first front shutter 1640 includes a first portion of a bore opening and second front shutter includes a second portion of the bore opening. One such embodiment is described below in conjunction with FIG. 19.

FIG. 19 schematically illustrates a front view of RT system 1600 and a bore opening 1900, according to various embodiments. In the embodiment illustrated in FIG. 19, first front shutter 1640 includes a first portion 1901 of a bore opening 1900 and second front shutter 1650 includes a second portion 1902 of bore opening 1900. Therefore, when first front shutter 1640 is in the first (closed) position and second front shutter 1650 is in the third (closed) position, bore opening 1900 is formed. In such an embodiment, when first front shutter 1640 is in the first position and second front shutter 1650 is in the third position, first portion 1691 and second portion 1692 are adjacent to each other and together form bore opening 1690.

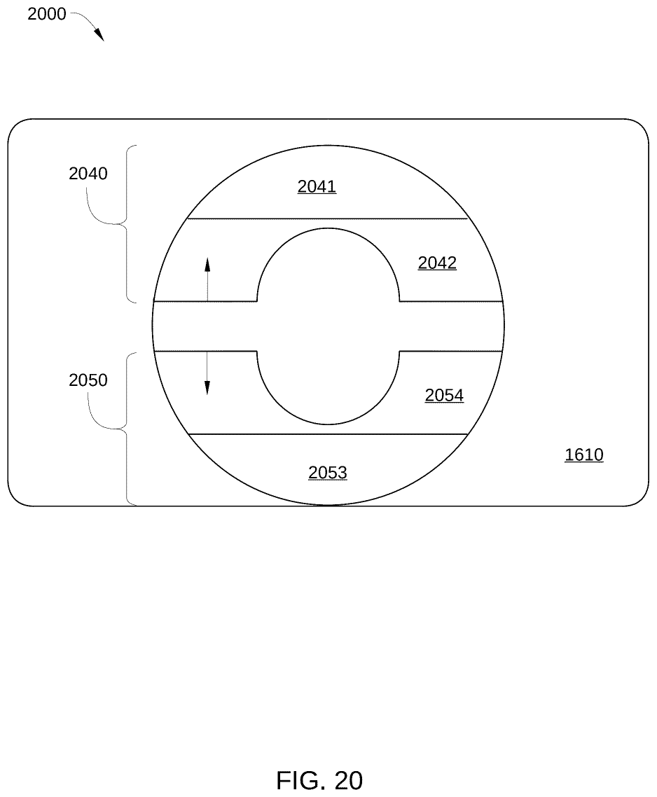

In the embodiment illustrated in FIGS. 16A and 16B, first front shutter 1640 moves horizontally between the first (closed) position and the second (open) position and the second front shutter 1650 moves horizontally between the third (closed) position and the fourth (open) position. In other embodiments, front shutters of a radiation therapy system move vertically between open and closed positions. One such embodiment is described below in conjunction with FIG. 20.

FIG. 20 schematically illustrates a front view of RT system 2000 with vertically actuated front shutters, according to various embodiments. In the embodiment illustrated in FIG. 20, RT system 2000 includes a first front shutter 2040 includes and second front shutter 2050, where first front shutter 2040 is implemented as an upper shutter and second front shutter 2050 is implemented as a lower shutter. As shown, first front shutter 2040 and second front shutter 2050 are in the process of opening vertically to enable access to a funnel-shaped cavity of RT system 2000. Thus, first front shutter 2040 moves vertically between a first (closed) position and a second (open) position and second front shutter 2050 moves vertically between a third (closed) position and a fourth (open) position.

In the embodiment illustrated in FIG. 20, first front shutter 2040 includes a first stage 2041 and a second stage 2042, and second front shutter 2050 includes a third stage 2053 and a fourth stage 2054. In other embodiments, first front shutter 1640 and/or second front shutter 1659 each include a single stage or three or more stages.

While various aspects and embodiments have been disclosed herein, other aspects and embodiments will be apparent to those skilled in the art without departing from the scope and spirit of the described embodiments. The various aspects and embodiments disclosed herein are for purposes of illustration and are not intended to be limiting, with the true scope and spirit being indicated by the following claims.

1. A radiation therapy system comprising:
a stationary support structure fixed to a support surface external to the radiation therapy system;
a rotatable gantry with a treatment-delivering radiation source and at least one X-ray imager mounted thereon, wherein the treatment-delivering radiation source directs a treatment beam through a treatment isocenter of the radiation therapy system and wherein the rotatable gantry is rotatably coupled to the support structure and is configured to rotate about a bore of the radiation therapy system; and
a couch-positioning system for rotating a couch about a center of rotation, wherein the couch-positioning system is rotatably coupled to the support surface and the center of rotation is vertically aligned with the treatment isocenter.

2. The radiation therapy system of claim 1, further comprising an imaging X-ray source that is mounted on the rotatable gantry and directs imaging X-rays through the treatment isocenter and toward the at least one X-ray imager.

3. The radiation therapy system of claim 1, wherein an axis of rotation of the rotatable gantry is parallel to a longitudinal axis of the bore.

4. The radiation therapy system of claim 1, wherein the bore comprises an open bore.

5. The radiation therapy system of claim 1, wherein the rotatable gantry includes a conical truss for supporting the treatment-delivering radiation source and the at least one X-ray imager.

6. The radiation therapy system of claim 5, wherein the conical truss has a center line that is congruent with an axis of rotation of the rotatable gantry.

7. The radiation therapy system of claim 5, wherein the conical truss comprises a truncated sheet metal cone.

8. The radiation therapy system of claim 5, wherein the conical truss forms a funnel-shaped cavity between the bore and the rotatable couch.

9. The radiation therapy system of claim 8, wherein the treatment isocenter is disposed within the funnel-shaped cavity.

10. The radiation therapy system of claim 8, wherein the funnel-shaped cavity is defined at least in part by a cone angle of the conical truss.

11. The radiation therapy system of claim 8, wherein the at least one X-ray imager is mounted on the rotatable gantry outside the funnel-shaped cavity.

12. The radiation therapy system of claim 11, wherein the at least one X-ray imager has an X-ray receiving surface that is oriented orthogonal to X-rays received by the at least one X-ray imager.

13. The radiation therapy system of claim 1, wherein the couch positioning system is configured to rotate the couch about the center of rotation to a maximum rotated position of the couch that is within a funnel-shaped cavity of the radiation therapy system.

14. The radiation therapy system of claim 13, wherein the maximum rotated position of the couch is based on the couch being positioned at a maximum lateral position.

15. The radiation therapy system of claim 1, wherein the couch positioning system comprises a turntable that is coupled to the couch and rotates about the center of rotation.

16. The radiation therapy system of claim 1, wherein the center of rotation is vertically aligned with a longitudinal axis of the bore.

17. The radiation therapy system of claim 1, wherein the at least one X-ray imager comprises an X-ray imager that acquires images based on the treatment beam and at least one additional X-ray imager that acquires images based on an imaging X-ray beam.

18. The radiation therapy system of claim 17, wherein the at least one additional X-ray imager comprises a first additional X-ray imager mounted on the rotatable gantry and a second additional X-ray imager mounted on the rotatable gantry.

19. The radiation therapy system of claim 17, further comprising an imaging X-ray source mounted on the rotatable gantry for generating the imaging X-ray beam.

20. The radiation therapy system of claim 19, wherein the imaging X-ray source mounted on the rotatable gantry for generating the imaging X-ray beam comprises a kilo-Volt X-ray source.
